(19) United States
(12) Patent Application Publication
(10) Pub. No.: US 2024/0182816 A1
(43) Pub. Date: Jun. 6, 2024

(54) TREATMENT COMPOSITION

(71) Applicant: The Procter & Gamble Company, Cincinnati, OH (US)

(72) Inventors: Mattia COLLU, Brussels (BE); Johan SMETS, Brussels (BE); Conny Erna Alice JOOS, Brussels (BE); Susana FERNANDEZ PRIETO, Castellon (ES); Maria Carolina ABREU MESTRE, Brussels (BE); Gaetano ALETTA, Brussels (BE); Linsheng FENG, Menasha, WI (US); Robert Stanley BOBNOCK, Menasha, WI (US)

(21) Appl. No.: 18/522,382

(22) Filed: Nov. 29, 2023

(30) Foreign Application Priority Data
Dec. 1, 2022 (GB) 2218059.0

(51) Int. Cl. C11D 3/22; A61K 8/73; A61Q 5/12; A61Q 19/10; C11D 1/02; C11D 3/50

(52) U.S. Cl. CPC C11D 3/222; A61K 8/736; A61Q 5/12; A61Q 19/10; C11D 1/02; C11D 3/50; A61K 2800/5424; A61K 2800/596

(57) ABSTRACT

A treatment composition that includes a treatment adjunct and a population of core/shell delivery particles, where the shells of the particles are made, in part, from biopolymers, and where the particles are characterized as having certain ductile properties. Related methods of making and using such compositions.

### FIELD OF THE INVENTION

The present disclosure relates to a treatment composition that includes a treatment adjunct and a population of core/shell delivery particles, where the shells of the particles are made, in part, from biopolymers, and where the particles are characterized as having certain ductile properties. The present disclosure also relates to related methods of making and using such compositions.

### BACKGROUND OF THE INVENTION

Delivery particles, particularly core/shell delivery particles, are a convenient way to deliver benefit agents in treatment compositions such as laundry products. For environmental reasons, it may be desirable to use delivery particles that have a shell made from naturally-derived and/or biodegradable materials, such as biopolymers.

Core/shell delivery particles are typically intended to have frangible characteristics. When intact, the particle shells protect benefit agents in the core for convenient delivery. Upon rupturing, the particles release the benefit agents.

Manufacturers of delivery particles have traditionally been faced with the challenge of forming populations of particles that rupture at a desired time or touchpoint. Much emphasis in the field has been placed on selecting materials and processing conditions that result in particles having a desired rupture profile. For example, core/shell delivery particles may be categorized, by their fracture strength and/or rupture stress, as such characteristics can be predictive of the conditions under which a particle is likely to release the benefit agent.

Despite the industry's emphasis on frangible capsules, it is believed that relying on rupture-for-release can have pitfalls. For example, despite the manufacturer's best intentions, the delivery particles may not rupture at the desired touchpoint(s). Additionally, given that the vast majority of the benefit agent is released only upon rupture, frangible capsules tend to have an all-or-nothing release profile, which may result in a user experiencing too little or too much of the benefit agent at any given point. These challenges can result in a suboptimal user/consumer experience.

There remains a need for treatment compositions that include core/shell delivery particles having improved or preferred release profiles. It is further preferred that such delivery particles be made, at least in part, from naturally-derived and/or biodegradable materials.

### SUMMARY OF THE INVENTION

The present disclosure relates to treatment compositions that include populations of delivery particles, where the delivery particles are characterized by certain ductile properties.

For example, the present disclosure relates to a treatment composition that includes a treatment adjunct and a population of delivery particles, where the delivery particles include a core and a shell surrounding the core, where the core includes a benefit agent, where the shell includes a polymeric material, where the polymeric material includes the reaction product of a biopolymer and a cross-linking agent, where the population of delivery particles is characterized by at least one, preferably at least two, more preferably all three, of the following: (a) a Volume-Weighted Ductile Energy that is greater than about 3.5, based on fifty delivery particles selected at random being compressed by a blunt probe moving at 2 μm/s; (b) at least about 30%, by number, of the delivery particles being characterized as Completely Ductile particles, based on fifty delivery particles selected at random being compressed by a blunt probe moving at 2 μm/s; (c) less than 35%, by number, of the delivery particles being characterized as Single Rupture particles, based on fifty delivery particles selected at random being compressed by a blunt probe moving at 2 μm/s.

The present disclosure also relates to a process of making a treatment composition according to the present disclosure, where the method includes the steps of: providing a base composition, where the base composition includes the treatment adjunct, and combining the population of delivery particles with the base composition.

The present disclosure also relates to a method of treating a surface, where the method includes the step of: contacting the surface, preferably a fabric, with a treatment composition according to the present disclosure.

### BRIEF DESCRIPTION OF THE DRAWINGS

The figures herein are illustrative in nature and are not intended to be limiting.

FIG. 1 shows the basic set-up for measuring the diameter of a delivery particle.

FIG. 2 shows compression curves (e.g., Speed-Depth and Load-Depth curves) for an exemplary delivery particle that ruptures.

FIG. 3 shows compression curves for a Completely Ductile particle.

FIG. 4 shows compression curves for a Single-Rupture particle.

FIG. 5 shows compression curves for a Multiple-Rupture particle.

FIG. 6 shows a Load-Depth curve for an illustrative Completely Ductile particle.

FIG. 7 shows a Load-Depth curve for an illustrative Single-Rupture particle.

FIG. 8 shows a Load-Depth curve for an illustrative Multiple-Rupture particle.

FIG. 9 shows a distribution of the measured Ductile Energy values of an illustrative population of delivery particles.

FIG. 10 shows a distribution of the Log (Ductile Energy) values of an illustrative population of delivery particles.

FIG. 11 shows a distribution of Rescaled Log (Ductile Energy) values of an illustrative population of delivery particles.

FIG. 12 shows a graph of particle size vs. Rescaled Log (Ductile Energy).

FIG. 13 shows a distribution of volume fractions by particle diameter of an illustrative population of delivery particles.

### DETAILED DESCRIPTION OF THE INVENTION

The present disclosure relates to treatment compositions that include benefit-agent-containing delivery particles having shells made, at least in part, from biopolymers. In contrast to the known, frangible behavior of previous core/shell particles, the delivery particles of the present disclosure are characterized by having desirable ductile properties. Particles having the ductility described herein can result in improved release profiles.

Without wishing to be bound by theory, it is believed that the delivery particles of the present disclosure have relatively flexible shells and are able to provide delivery or release profiles that are different, and in at least some cases more desired, than particles characterized by release via rupture. For example, it is believed that due to having relatively flexible shells, the particles of the present disclosure, at least at the population level, are more likely to survive treatment processes that include physical agitation, such as washing and/or drying processes in automatic laundry machines. Further, the ductile particles of the disclosed particle populations are not characterized by the all-or-nothing release profiles of their rupturable counterparts, thereby providing improved performance at one or more touchpoints.

It is believed that the ductile characteristics of the present populations of delivery particles can be provided, and even tuned, by the selection of certain materials, starting amounts, and/or processing conditions. For example, it is believed that biopolymers according to the present disclosure, such as chitosan, provide a useful starting material for the formation of ductile particle shells. For example, it is believed that certain biopolymers are characterized by a desirable water-holding capacity, e.g., due to the presence of hydroxyl groups, and may swell and/or increase in elasticity in the presence of water. As an added benefit, the biopolymers of the present disclosure are naturally-derived and/or biodegradable, thereby improving the environmental footprint of the present delivery particles.

The delivery particles, treatment compositions, and related methods of the present disclosure are discussed in more detail below.

As used herein, the articles “a” and “an” when used in a claim, are understood to mean one or more of what is claimed or described. As used herein, the terms “include,” “includes,” and “including” are meant to be non-limiting. The compositions of the present disclosure can comprise, consist essentially of, or consist of, the components of the present disclosure.

The terms “substantially free of” or “substantially free from” may be used herein. This means that the indicated material is at the very minimum not deliberately added to the composition to form part of it, or, preferably, is not present at analytically detectable levels. It is meant to include compositions whereby the indicated material is present only as an impurity in one of the other materials deliberately included. The indicated material may be present, if at all, at a level of less than 1%, or less than 0.1%, or less than 0.01%, or even 0%, by weight of the composition.

As used herein “consumer product,” means baby care, beauty care, fabric & home care, family care, feminine care, and/or health care products or devices intended to be used or consumed in the form in which it is sold, and not intended for subsequent commercial manufacture or modification. Such products include but are not limited to diapers, bibs, wipes; products for and/or methods relating to treating human hair, including bleaching, coloring, dyeing, conditioning, shampooing, styling, leave-on treatments, and boosters; deodorants and antiperspirants; personal cleansing; skin care including application of creams, lotions, and other topically applied products for consumer use; and shaving products, products for and/or methods relating to treating fabrics, hard surfaces and any other surfaces in the area of fabric and home care, including: air care, car care, dishwashing, fabric conditioning (including softening), laundry detergency, laundry and rinse additive and/or care, hard surface cleaning and/or treatment, and other cleaning for consumer or institutional use; products and/or methods relating to bath tissue, facial tissue, paper handkerchiefs, and/or paper towels; tampons, feminine napkins; adult incontinence products; products and/or methods relating to oral care including toothpastes, tooth gels, tooth rinses, denture adhesives, tooth whitening; over-the-counter health care including cough and cold remedies; pest control products; and water purification.

As used herein the phrase “fabric care composition” includes compositions and formulations designed for treating fabric. Such compositions include but are not limited to, laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, post-rinse fabric treatment, ironing aid, unit dose formulation, delayed delivery formulation, detergent contained on or in a porous substrate or nonwoven sheet, and other suitable forms that may be apparent to one skilled in the art in view of the teachings herein. Such compositions may be used as a pre-laundering treatment, a post-laundering treatment, or may be added during the rinse or wash cycle of the laundering operation.

As used herein, “delivery particles,” “particles,” “encapsulates,” “microcapsules,” and “capsules” are used interchangeably, unless indicated otherwise. As used herein, these terms typically refer to core/shell delivery particles.

Unless otherwise noted, all component or composition levels are in reference to the active portion of that component or composition, and are exclusive of impurities, for example, residual solvents or by-products, which may be present in commercially available sources of such components or compositions.

All temperatures herein are in degrees Celsius (C) unless otherwise indicated. Unless otherwise specified, all measurements herein are conducted at 20° C. and under the atmospheric pressure.

In all embodiments of the present disclosure, all percentages are by weight of the total composition, unless specifically stated otherwise. All ratios are weight ratios, unless specifically stated otherwise.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Treatment Composition

The present disclosure relates to treatment compositions (or simply “compositions” as used herein). The compositions of the present disclosure may comprise a treatment adjunct and a population of delivery particles, each described in more detail below. The treatment compositions may be useful in the methods of treating surfaces, such as fabrics, described herein.

The treatment composition is preferably a consumer product composition. The consumer products compositions of the present disclosure may be useful in baby care, beauty care, fabric care, home care, family care, feminine care, and/or health care applications. The consumer product compositions may be useful for treating a surface, such as fabric, hair, or skin. The consumer product compositions may be intended to be used or consumed in the form in which it is sold. The consumer product compositions of the present disclosure are typically not intended for subsequent commercial manufacture or modification.

The consumer product composition may preferably be a fabric care composition, a hard surface cleaner composition, a dish care composition, a hair care composition (such as shampoo or conditioner), a body cleansing composition, or a mixture thereof, preferably a fabric care composition.

The consumer product composition may be a fabric care composition, such as a laundry detergent composition (including a heavy-duty liquid washing detergent or a unit dose article), a fabric conditioning composition (including a liquid fabric softening and/or enhancing composition), a laundry additive, a fabric pre-treat composition (including a spray, a pourable liquid, or a spray), a fabric refresher composition (including a spray), or a mixture thereof. The treatment composition is preferably a fabric conditioning composition, even more preferably a liquid fabric conditioning composition.

The composition may be a beauty care composition, such as a hair treatment product (including shampoo and/or conditioner), a skin care product (including a cream, lotion, or other topically applied product for consumer use), a shave care product (including a shaving lotion, foam, or pre- or post-shave treatment), personal cleansing product (including a liquid body wash, a liquid hand soap, and/or a bar soap), a deodorant and/or antiperspirant, or mixtures thereof.

The composition may be a home care composition, such as an air care, car care, dishwashing, hard surface cleaning and/or treatment, and other cleaning for consumer or institutional use.

The treatment composition may be in the form of a liquid composition, a granular composition, a hydrocolloid, a single-compartment pouch, a multi-compartment pouch, a dissolvable sheet, a pastille or bead, a fibrous article, a tablet, a stick, a bar, a flake, a foam/mousse, a non-woven sheet, or a mixture thereof.

The treatment composition may be in the form of a liquid. The liquid composition may preferably include from about 50% to about 97%, preferably from about 60% to about 96%, more preferably from about 70% to about 95%, or even from about 80% to about 95%, by weight of the fabric treatment composition, of water. The liquid composition may be a liquid fabric conditioner. The liquid may be packaged in a pourable bottle. The liquid may be packaged in an aerosol can or other spray bottle. Suitable containers are described in more detail below.

The treatment composition may be in the form of a solid. The composition may be in the form of a bead or pastille, which may be pastilled from a liquid melt. The composition may be an extruded product. The treatment composition may be in the form of a powder or granules.

The composition may be in the form of a unitized dose article, such as a tablet, a pouch, a sheet, or a fibrous article. Such pouches typically include a water-soluble film, such as a polyvinyl alcohol water-soluble film, that at least partially encapsulates a composition. Suitable films are available from MonoSol, LLC (Indiana, USA). The composition can be encapsulated in a single or multi-compartment pouch. A multi-compartment pouch may have at least two, at least three, or at least four compartments. A multi-compartmented pouch may include compartments that are side-by-side and/or superposed. The composition contained in the pouch or compartments thereof may be liquid, solid (such as powders), or combinations thereof. Pouched compositions may have relatively low amounts of water, for example less than about 20%, or less than about 15%, or less than about 12%, or less than about 10%, or less than about 8%, by weight of the detergent composition, of water. The composition may comprise 0% water, or at least 0.1% water, or at least 1% water.

The treatment composition may be in the form of a spray and may be dispensed, for example, from a bottle via a trigger sprayer and/or an aerosol container with a valve.

The treatment composition may have a viscosity of from 1 to 1500 centipoises (1-1500 mPa*s), from 100 to 1000 centipoises (100-1000 mPa*s), or from 200 to 500 centipoises (200-500 mPa*s) at 20 s⁻¹ and 21° C.

The treatment compositions of the present disclosure may be characterized by a pH of from about 2 to about 12, or from about 2 to about 8.5, or from about 2 to about 7, or from about 2 to about 5. The treatment compositions of the present disclosure may have a pH of from about 2 to about 4, preferably a pH of from about 2 to about 3.7, more preferably a pH from about 2 to about 3.5, preferably in the form of an aqueous liquid. It is believed that such pH levels facilitate stability of the quaternary ammonium ester compound, when present. On the other hand, traditional detergent compositions are typically characterized by a pH of from about 7 to about 12, preferably from about 7.5 to about 11. In some cases, acidic detergents may be desirable and may be characterized by a pH of from about 2 to about 6, preferably from about 2 to about 4. Compositions useful for certain beauty care applications, such as skin creams and/or shampoos, may be characterized by a pH of from about 4 to about 7, preferably from about 5 to about 6. The pH of a composition is determined by dissolving/dispersing the composition in deionized water to form a solution at 10% concentration, at about 20° C.

Additional components and/or features of the compositions are discussed in more detail below.

### Population of Delivery Particles

The treatment compositions of the present disclosure comprise a population of delivery particles. The delivery particles comprise a core and a shell surrounding the core. The core comprises a benefit agent (preferably a fragrance material), and optionally a partitioning modifier. The core can be a liquid or a solid, preferably a liquid, at room temperature. The shell comprises a polymeric material, which is typically a reaction product of a biopolymer and a cross-linking agent.

The delivery particles of the present disclosure can be described as having ductile properties, at least at the population level. As generally used herein, “ductile” particles (or those having “ductility”) are those that can be deformed without material failure or rupture. Without wishing to be bound by theory, it is believed that the shells of the presently described particles are relatively flexible or pliable, while still being suitably robust to generally contain the benefit agents in the core. It is believed that instead of releasing the benefit agent by a rupture mechanism, the ductile particles of the present disclosure release the encapsulated benefit agent when squeezed or otherwise deformed without breaking. This may result in a longer-lasting release profile because the benefit agent is not necessarily released in a single rupture event. Additionally or alternatively, relatively low forces may be required to obtain a release of the encapsulated benefit agent, as complete rupture of the particle is not required for a release event.

As described in more detail below, it is believed that the relative ductility of the presently described particle populations can be influenced by the selection of certain starting materials, starting amounts, and/or processing conditions. For example, the use of certain starting materials and amounts and/or ratios thereof, particle size and/or shell thickness, the use and amount of a partitioning modifier, and/or the use of certain pH or milling temperatures during particle formation can be leveraged to provide populations of delivery particles that have desirable ductility and performance characteristics.

The population of delivery particles of the present disclosure may be characterized by a Volume-Weighted Ductile Energy greater than about 3.5, preferably greater than about 3.8, based on fifty delivery particles selected at random being compressed by a blunt probe moving at 2 μm/s. The population of delivery particles of the present disclosure may be characterized by a Volume-Weighted Ductile Energy of from about 3.5 to about 10.0, preferably from about 3.5 to about 7.5, more preferably from about 3.8 to about 6.0, more preferably from about 4.0 to about 5.5, even more preferably from about 4.5 to about 5.2, based on fifty delivery particles selected at random being compressed by a blunt probe moving at 2 μm/s.

It is believed that populations of delivery particles characterized Volume-Weighted Ductile Energy at the described levels provide desirable performance at certain touchpoints, such as Dry Fabric Odor, and/or may be well distributed over time, providing longevity and consistency benefits. Such populations may also be useful in certain applications, particularly where there is high shear in usage, such as through the wash and/or rinse cycle of an automatic laundry machine. At lower levels of Volume-Weighted Ductile Energy, the particles may not display sufficient ductility to provide the desired benefits; for example, they may collapse too early during manufacture, transport, or usage (e.g., during a wash cycle) and prematurely release the encapsulated benefit agent. More details on how to determine the Volume-Weighted Ductile Energy of a population of delivery particles can be found in the Test Methods section below.

When the treatment composition is expected or intended to be used under relatively high shear conditions, it may be preferred to use populations of delivery particles that are characterized by a relatively higher Volume-Weighted Ductile Energy. For example, treatment compositions that are intended to be used in the wash cycle of an automatic laundry machine (such as heavy-duty liquid detergents, unitized dose detergents, and/or laundry additives for the wash cycle, such as scent beads) may comprise populations of delivery particles that are characterized by a Volume-Weighted Ductile Energy of from about 4.5 to about 6.0, preferably from about 5.0 to about 5.5, based on fifty delivery particles selected at random being compressed by a blunt probe moving at 2 μm/s. It is believed that such particles can better withstand the high shear conditions while maintaining an adequate amount of the benefit agent in the core.

When the treatment composition is expected or intended to be used under relatively lower shear conditions, it may be preferred to use populations of delivery particles that are characterized by a relatively lower Volume-Weighted Ductile Energy. For example, treatment compositions that are intended to be used in the rinse cycle of an automatic laundry machine (such as liquid fabric enhancers) may comprise populations of delivery particles that are characterized by a Volume-Weighted Ductile Energy of from about 3.5 to about 5, preferably from about 3.8 to about 4.8, more preferably from about 4.0 to about 4.5, based on fifty delivery particles selected at random being compressed by a blunt probe moving at 2 μm/s. It is believed that such particles can withstand the lower shear conditions while maintaining an adequate amount of the benefit agent in the core and offering a convenient release of the benefit agent.

It may be desirable to vary the Volume-Weighted Ductile Energy of the particle population as the particle size varies. For example, populations having relatively larger particles may be characterized by relatively larger Volume-Weighted Ductile Energy. Without wishing to be bound by theory, it is believed that larger particles typically have more mass and therefore can absorb more energy. Thus, the size and Volume-Weighted Ductile Energy of the particle population may be selected for certain applications and/or release profiles. For example, when the population of delivery particles is characterized by a volume-weighted median particle size of from about 20 to about 40 microns, the Volume-Weighted Ductile Energy of the population may preferably be from about 3.5 to about 7.5, more preferably from about 4.5 to about 6.0. When the population of delivery particles is characterized by a volume-weighted median particle size of from about 10 to about 25 microns, the Volume-Weighted Ductile Energy of the population may preferably be from about 3.0 to about 5.0, more preferably from about 3.5 to about 4.0, based on fifty delivery particles selected at random being compressed by a blunt probe moving at 2 μm/s.

Any single delivery particle can be categorized as a Completely Ductile particle, a Single Rupture particle, or a Multiple Rupture particle upon being compressed by a blunt probe moving at 2 μm/s , as described in more detail in the Test Methods section. By sampling a particular number of particles, for example fifty particles, a population of delivery particles can be described in terms of the proportion of particles (for example, as a percentage by number) that fall into any one or more of those categories. The relative proportion(s) can give an indication of the population's behavior as a whole, which can help predict the relative performance of the population in treatment compositions. When categorizing the population of delivery particles using these categories, the respective percentages of Completely Ductile particles, Single Rupture particles, and Multiple Rupture particles will typically add up to 100%.

The population of delivery particles of the present disclosure may be characterized by at least about 30%, preferably at least about 50%, by number, of the delivery particles being characterized as Completely Ductile particles, based on fifty delivery particles selected at random being compressed by a blunt probe moving at 2 μm/s. It is believed that a population having a relatively high proportion of Completely Ductile particles can provide desirable performance. Without wishing to be bound by theory, it is believed that because the release of the encapsulated benefit agent is not concentrated at a single point in time (e.g., a rupture event), the release of benefit agent from the particles of the present disclosure tends to be more gradual and/or linear over time. More details on how to determine the relative proportion of Completely Ductile particles in a population can be found in the Test Methods section below.

The population of delivery particles of the present disclosure may be characterized by less than 35%, preferably less than 25%, by number, of the delivery particles being characterized as Single Rupture particles, based on fifty delivery particles selected at random being compressed by a blunt probe moving at 2 μm/s. It is believed that a population having a relatively low proportion of Completely Ductile particles can provide desirable performance. More details on how to determine the relative proportion of Single Rupture particles in a population can be found in the Test Methods section below.

The population of delivery particles of the present disclosure may comprise delivery particles that are characterized as Multiple Rupture particles, based on fifty delivery particles selected at random being compressed by a blunt probe moving at 2 μm/s. More details on how to determine the relative proportion of Single Rupture particles in a population can be found in the Test Methods section below.

It is preferred that the population of delivery particles is characterized by at least one, preferably at least two, more preferably all three, of the following, where each is based on fifty delivery particles selected at random being compressed by a blunt probe moving at 2 μm/s according to the test methods described below: (a) a Volume-Weighted Ductile Energy greater than about 3.5; (b) at least about 30%, by number, of the delivery particles are characterized as Completely Ductile particles; (c) less than 35%, by number, of the delivery particles are characterized as Single Rupture particles.

The treatment composition may comprise from about 0.05% to about 20%, or from about 0.05% to about 10%, or from about 0.1% to about 5%, or from about 0.2% to about 2%, by weight of the composition, of delivery particles. The composition may comprise a sufficient amount of delivery particles to provide from about 0.05% to about 10%, or from about 0.1% to about 5%, or from about 0.1% to about 2%, by weight of the composition, of the encapsulated benefit agent, which may preferably be fragrance material having one or more perfume raw materials, to the composition. When discussing herein the amount or weight percentage of the delivery particles, it is meant the sum of the wall material and the core material.

The population of delivery particles according to the present disclosure may be characterized by a volume-weighted median particle size from about 1 to about 100 microns, preferably from about 10 to about 100 microns, preferably from about 15 to about 50 microns, more preferably from about 20 to about 40 microns, even more preferably from about 25 to about 35 microns. For certain compositions, it may be preferred that the population of delivery particles is characterized by a volume-weighted median particle size from about 1 to about 50 microns, preferably from about 5 to about 20 microns, more preferably from about 10 to about 15 microns. Different particle sizes are obtainable by controlling droplet size during emulsification.

The delivery particles may be characterized by a ratio of core to shell up to 99:1, or even 99.5:0.5, on the basis of weight. The shell may be present at a level of from about 1% to about 25%, preferably from about 1% to about 20%, preferably from about 1% to 15%, more preferably from about 5% to about 15%, even more preferably from about 10% to about 15%, even more preferably from about 10% to about 12%, by weight of the delivery particle. The shell may be present at a level of least 1%, preferably at least 3%, more preferably at least 5% by weight of the delivery particle. The shell may be present at a level of up to about 20%, preferably up to about 15%, more preferably up to about 12%, by weight of the delivery particle.

The delivery particles may be cationic in nature, preferably cationic at a pH of 4.5. The delivery particles may be characterized by a zeta potential of at least 15 millivolts (mV) at a pH of 4.5. The delivery particles can be fashioned to have a zeta potential of at least 15 millivolts (mV) at a pH of 4.5, or even at least 40 mV at a pH of 4.5, or even at least 60 mV at a pH of 4.5. Polyurea capsules prepared with chitosan typically exhibit positive zeta potentials. Such capsules have improved deposition efficiency on fabrics. At higher pH, the particles may be able to be made nonionic or anionic.

The delivery particles of the present disclosure comprise a shell surrounding a core. (As used herein, “shell” and “wall” are used interchangeably with regard to the delivery particles, unless indicated otherwise.) The shell comprises a polymeric material. The polymeric material comprises, and preferably is, the reaction product of a biopolymer and a cross-linking agent.

The biopolymer may preferably be selected from the group consisting of a polysaccharide, a protein, a nucleic acid, a polyphenolic compound, derivatives thereof, and combinations thereof. Preferably, the biopolymer is selected from the group consisting of:
(a) a polysaccharide selected from the group consisting of chitosan, starch, modified starch, dextran, maltodextrin, dextrin, cellulose, modified cellulose, hemicellulose, chitin, alginate, lignin, gum, pectin, fructan, carrageenan, agar, pullulan, suberin, cutin, cutan, melanin, silk fibronin, derivatives thereof, and combinations thereof;
(b) a protein selected from the group consisting of gelatin, collagen, casein, sericin, fibroin, whey protein, zein, soy protein, plant storage protein (plant protein isolate, plant protein concentrate), gluten, peptide, actin, derivatives thereof, and combinations thereof;
(c) a nucleic acid selected from the group consisting of polynucleotides, RNA, DNA, derivatives thereof, and combinations thereof;
(d) a polyphenolic compound selected from the group consisting of tannins, lignans, derivatives thereof, and combinations thereof; or
(e) combinations thereof.

The biopolymer preferably comprises primary amine groups. The primary amine groups can react with the cross-linking agents, preferably polyisocyanates, to form the polymeric material, which may be described as a cross-linked biopolymer.

Amine-containing biopolymers, such as amine-containing or amine-modified saccharides, may be preferred, for example due to convenient availability, biodegradability, and/or performance reasons. A particularly preferred material is chitosan. Thus, the biopolymer may preferably be chitosan, a derivative thereof, or a combination thereof. Preferably, the biopolymer is acid-treated chitosan, redox-initiator-treated chitosan, a derivative thereof, or a combination thereof.

The chitosan may preferably be acid-treated chitosan. For example, chitosan (which, prior to acid treatment, may be referred to as raw chitosan or parent chitosan) may preferably be treated at a pH of 6.5 or less with an acid for at least one hour, preferably from about one hour to about three hours, at a temperature of from about 25° C. to about 99° C., preferably from about 75° C. to about 95° C. The acid may be selected from a strong acid (such as hydrochloric acid), an organic acid (such as formic acid or acetic acid), or a mixture thereof. The chitosan may preferably be acid-treated at a pH of from 2 to 6.5, or even from a pH of from 4 to 6.

The chitosan may be treated with a redox initiator (e.g., a redox-initiator-treated chitosan). For example, a redox initiator, preferably comprising a persulfate or a peroxide, may be added to the water phase and/or to the emulsion when forming the delivery particles. A redox initiator, which may comprise a persulfate or a peroxide, can be added to the acid-treated chitosan. In an in situ variation, a redox initiator can be added to the emulsion following combining of the oil phase and water phase under high shear agitation. The redox initiator advantageously depolymerizes the hydrolyzed chitosan or modified chitosan reducing viscosity facilitating polymer formation of the shell in the capsule formation process. Modification of chitosan with the epoxide, aldehyde, or α,β-unsaturated compound is preferably accomplished prior to addition of the redox initiator, although the redox initiator (peroxide or persulfate) can be introduced at the same time as the modifying compound or even before. The redox initiator can be selected from the group consisting of ammonium persulfate, sodium persulfate, potassium persulfate cesium persulfate, benzoyl peroxide, hydrogen peroxide, and mixtures thereof. The redox initiator, preferably a persulfate or peroxide, may be present at a level of from about 0.1 wt % to about 99 wt % of the chitosan.

The biopolymer, preferably chitosan, more preferably acid-treated chitosan, may preferably be characterized by a molecular weight of from about 1 kDal to about 1000 kDal, preferably from about 50 kDal to about 600 kDal, more preferably from about 100 kDal to about 500 kDal, even more preferably from about 100 kDal to about 300 kDal, even more preferably from about 100 kDal to about 200 kDal. Without wishing to be bound by theory, it is believed that biopolymers characterized by a relatively low molecular weight are less effective at forming suitable delivery particles, while those having relatively high molecular weights tend to be difficult to process. The method used to determine the chitosan's molecular weight and related parameters is provided in the Test Methods section below and uses gel permeation chromatograph with multi-angle light scatter and refractive index detection (GPC-MALS/RI) techniques.

The chitosan, when present, may be characterized by a degree of deacetylation of at least 50%, preferably from about 50% to about 99%, more preferably from about 75% to about 90%, even more preferably from about 80% to about 85%. The degree of deacetylation can affect the solubility of the chitosan, which in turn can affect its reactivity or behavior in the process of forming the particle shells. For example, a degree of deacetylation that is too low (e.g., below 50%) results in chitosan that is relatively insoluble and relatively unreactive. A degree of deacetylation that is relatively high can result in chitosan that is very soluble, resulting in relatively little of it traveling to the oil/water interface during shell formation.

The chitosan, when present, may comprise anionically modified chitosan, cationically modified chitosan, or a combination thereof. Modifying the chitosan in an anionic and/or cationic fashion can change the character of the shell of the delivery particle, for example, by changing the surface charge and/or zeta potential, which can affect the deposition efficiency and/or formulation compatibility of the particles.

As mentioned above, the shell is a polymeric material that is the reaction product of the biopolymer chitosan and a cross-linking agent. The cross-linking material is preferably a material selected from the group consisting of a polyisocyanate, a polyacrylate, a poly(meth)acrylate, a polyisothiocyanate, an aldehyde, an epoxy compound, a polyphenol, a carbonyl halide, an aziridine, and combinations thereof. The cross-linking agent is more preferably selected from the group consisting of a polyisocyanate, an epoxy compound, a bifunctional aldehyde, and combinations thereof.

The cross-linking agent is preferably a polyisocyanate, particularly when the biopolymer comprises amine groups. It is believed that such materials favorable react with the amine groups of the biopolymer to form effective, cross-linked polymeric walls. The polymeric material of the shells may preferably comprise a polyurea resin, which resin may comprise the reaction product of a polyisocyanate and a chitosan.

The polyisocyanate material useful in the present disclosure is to be understood for purposes hereof as isocyanate monomer, isocyanate oligomer, isocyanate prepolymer, or dimer or trimer of an aliphatic or aromatic isocyanate. By “polyisocyanate,” it is intended to mean a material or compound that includes two or more isocyanate moieties. All such monomers, prepolymers, oligomers, or dimers or trimers of aliphatic or aromatic isocyanates are intended encompassed by the term “polyisocyanate” herein. The polyisocyanates useful in the present disclosure comprise isocyanate monomers, oligomers or prepolymers, or dimers or trimers thereof, having at least two isocyanate groups. Preferred cross-linking can be achieved with polyisocyanates having at least three functional groups.

Aromatic polyisocyanates may be preferred; however, aliphatic polyisocyanates and blends thereof may be useful. Aliphatic polyisocyanate is understood as a polyisocyanate which does not comprise any aromatic moiety. Aromatic polyisocyanate is understood as a polyisocyanate which comprises at least one aromatic moiety. The cross-linking agent may comprise a mixture of an aromatic polyisocyanate and an aliphatic polyisocyanate.

The polyisocyanate, when aromatic, can be, but is not limited to, methylene diphenyl isocyanate, toluene diisocyanate, tetramethylxylidene diisocyanate, polyisocyanurate of toluene diisocyanate (commercially available from Bayer under the tradename Desmodur® RC), trimethylol propane-adduct of toluene diisocyanate (commercially available from Bayer under the tradename Desmodur® L75), or naphthalene-1,5-diisocyanate, phenylene diisocyanate, or trimethylol propane-adduct of xylylene diisocyanate (commercially available from Mitsui Chemicals under the tradename Takenate® D-110N).

Aliphatic polyisocyanates may include a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100).

Derivatives of polyisocyanates may include oligomers or polymers of isocyanate monomers. As a non-limiting example, the polyisocyanate may preferably comprise an oligomer or polymer of diphenylmethane diisocyanate (MDI), such as Mondur® MR-Light.

The polyisocyanate may preferably be selected from the group consisting of: a polyisocyanurate of toluene diisocyanate; a trimethylol propane adduct of toluene diisocyanate; a trimethylol propane adduct of xylylene diisocyanate; 2,2′-methylenediphenyl diisocyanate; 4,4′-methylenediphenyl diisocyanate; 2,4′-methylenediphenyl diisocyanate; [diisocyanato(phenyl)methyl]benzene; toluene diisocyanate; tetramethylxylidene diisocyanate; naphthalene-1,5-diisocyanate; 1,4-phenylene diisocyanate; 1,3-diisocyanatobenzene; derivatives thereof (such as pre-polymers, oligomers, and/or polymers thereof); and combinations thereof.

The particle shell may also be reinforced using additional co-crosslinkers such as multifunctional amines and/or polyamines, such as diethylene triamine (DETA), polyethylene imine, polyvinyl amine, or mixtures thereof. Acrylates may also be used as additional co-crosslinkers, for example to reinforce the shell.

The polymeric material may be formed in a reaction, where the weight ratio of the biopolymer, preferably a polysaccharide, more preferably chitosan or a derivative thereof (which can include acid-treated chitosan) present in the reaction to the cross-linker present in the reaction is from about 1:10 to about 10:1, preferably from about 1:5 to about 5:1, preferably from about 1:4 to about 5:1, more preferably from about 1:1 to about 5:1, more preferably from about 3:1 to about 5:1. It is believed that selecting desirable ratios of the biopolymer to the cross-linking agent can provide desired ductility benefits, as well as improved biodegradability.

It may be preferred that at least 21 wt % of the shell is comprised of moieties derived from the biopolymer, preferably from chitosan, more preferably from acid-treated chitosan. Biopolymer, preferably chitosan or a derivative thereof, as a percentage by weight of the shell may be from about 21% up to about 95% of the shell. The ratio of biopolymer, preferably chitosan, in the water phase as compared to the cross-linking agent, preferably polyisocyanate, in the oil phase may be, based on weight, from 21:79 (1:3.7) to 90:10 (1:0.11), or even from 33.3:66.6 (1:2) to 90:10 (9:1), or even from 50:50 (1:1) to 87.5:12.5 (7:1). The shell may comprise the biopolymer, preferably chitosan, at a level of 21 wt % or even greater, preferably from about 21 wt % to about 90 wt %, or even from 21 wt % to 85 wt %, or even 21 wt % to 75 wt %, or 21 wt % to 55 wt % of the total shell being biopolymer, preferably chitosan. The chitosan of this paragraph may preferably be acid-treated chitosan.

The reaction product that forms at least a part of the polymeric material may be formed in a reaction in which the biopolymer is initially present in an aqueous phase, and the cross-linking agent is initially present in an oil phase. The cross-linking agent is preferably present in the oil phase at a level of from about 1% to about 20%, preferably from about 2% to about 10%, more preferably from about 2.5% to about 5%, by weight of the oil phase. The cross-linking agent may be a polyisocyanate that is present in the oil phase at a level of greater than 1%, preferably 1.3%, preferably greater than 2%, more preferably greater than 2.5%, even more preferably greater than 2.9%.

The population of delivery particles may be made according to a process that comprises the following steps: (a) forming a water phase that includes chitosan as described herein, preferably where the water phase is at a pH of 6.5 or less, more preferably at a pH of from 3 to 6, and a temperature of at least 25° C.; (b) forming an oil phase that comprises at least one benefit agent, preferably fragrance material, and at least cross-linking agent, preferably at one polyisocyanate, and optionally a partitioning modifier; (c) forming an emulsion, preferably an oil-in-water emulsion, by mixing the water phase and the oil phase under high shear agitation, optionally adjusting the pH of the emulsion to be in a range of from pH 2 to pH 6; (d) curing the emulsion by heating, preferably to at least 40° C., for a time sufficient to form a shell at the interface of the oil droplets with the water phase, where the shell will comprise a polymeric material that is the reaction product of the chitosan and the cross-linking agent, and where the shell surrounds a core that comprises the benefit agent.

The population of delivery particles may be made according to a process that comprises the following steps: (a) forming a water phase by treating the chitosan with a mixture of a first acid and a second acid, the first acid comprising a strong acid, and the second acid comprising a weak acid, wherein the chitosan is treated at a pH of 6.5 or less, or even less than pH 6.5, or even at a pH of from 3 to 6, and a temperature of at least 25° C. for at least one hour, or for a period of time required to obtain a chitosan solution viscosity of not more than about 1500 cps of the acid-treated chitosan, or even not more than 500 cps; (b) forming an oil phase comprising dissolving together at least one benefit agent and at least one polyisocyanate, optionally with an added oil (e.g., partitioning modifier) and/or solvent; (c) forming an emulsion by mixing under high shear agitation the water phase and the oil phase into an excess of the water phase, thereby forming droplets of the oil phase and benefit agent dispersed in the water phase, and optionally adjusting the pH of the emulsion to be in a range from pH 2 to pH 6; (d) curing the emulsion by heating to at least 40° C., for a time sufficient to form a shell at an interface of the droplets with the water phase, the shell comprising the reaction product of the polyisocyanate and the acid treated chitosan, and the shell surrounding the core comprising the droplets of the oil phase and benefit agent.

Chitosan may be added into water in a jacketed reactor and at pH from 2 or even from 3 to 6.5, adjusted using acid such as concentrated HCl. The chitosan of this mixture may be acid-treated by heating to elevated temperature, such as 85° C. in 60 minutes, and then may be held at this temperature from 1 minute to 1440 minutes, or even longer. The water phase then may be cooled to 25° C. Optionally, deacetylating may also be further facilitated or enhanced by enzymes to depolymerize or deacetylate the chitosan. An oil phase may be prepared by dissolving an isocyanate such as trimers of xylylene Diisocyanate (XDI) or polymers of methylene diphenyl isocyanate (MDI), in oil at 25° C. The oil phase may then be added into the water phase and milled at high speed to obtain a targeted size. The emulsion may then be cured in one or more heating steps, such as heating to 40° C. in 30 minutes and holding at 40° C. for 60 minutes. Times and temperatures are approximate. The temperature and time are selected to be sufficient to form and cure a shell at the interface of the droplets of the oil phase with the water continuous phase. For example, the emulsion may be heated to 85° C. in 60 minutes and then held at 85° C. for 360 minutes to cure the particles. The slurry may then be cooled to room temperature.

To form a suitably ductile population of capsules, it may be preferred to adjust the pH of the water phase and/or the emulsion to greater than or equal to about 5.2, preferably greater than or equal to about 5.6, and up to about 6.5, preferably up to about 6. It is believed that the pH during particle shell formation can affect the ultimate ductility of the particle population.

To form a suitably ductile population of capsules, it may be preferred to make the capsules by a process that includes at least one milling step, which may preferably occur at a particular temperature. For example, the at least one milling step may occur at a temperature of at least about 7° C., 15° C., preferably at least about 20° C., more preferably at least about 25° C., even more preferably from about 25° C. to about 35° C. Milling may occur until the desired particle size is achieved. It is believed that the temperature during the milling step affect the ultimate ductility of the particle population.

The shell may degrade at least 50% after 20 days (or less) when tested according to test method OECD 301B. The shell may degrade at least 60% of its mass after 60 days (or less) when tested according to test method OECD 301B. The shell may preferably degrade at least 60% of its mass after 60 days (or less) when tested according to test method OECD 301B. The shell may degrade from 30-100%, preferably 40-100%, 50-100%, 60-100%, or 60-95%, in 60 days, preferably 50 days, more preferably 40 days, more preferably 28 days, more preferably 14 days.

The delivery particles of the present disclosure include a core. The core comprises a benefit agent, preferably a fragrance material. The core optionally comprises a partitioning modifier.

The core of a particle is surrounded by the shell. When ductile particles are compressed or otherwise deformed, it is believed that the benefit agent, preferably a fragrance material, in the core exits the particle by being squeezed through the shell. Additionally or alternatively, at least some of the benefit agent may diffuse through the shell. Even when ductile particles are present in populations of the present disclosure, some of the particles may rupture upon compression or deformation, resulting in release of the benefit agent. Suitable benefit agents located in the core may include benefit agents, such as suitable fragrance materials, that provide benefits to a surface, such as a fabric or hair.

The core may comprise from about 5% to about 100%, by weight of the core, of a benefit agent, preferably a fragrance material. The core may comprise from about 45% to about 95%, preferably from about 50% to about 80%, more preferably from about 50% to about 70%, by weight of the core, of the benefit agent, preferably a fragrance material.

The benefit agent in the core may be relatively hydrophobic. Such agents are compatible with the oil phases that are common in making the delivery particles of the present disclosure.

The benefit agent is selected so as to provide a benefit under preferred uses of the treatment composition. The benefit agent in the core may be selected from the group consisting of fragrance materials, silicone oils, waxes, hydrocarbons, higher fatty acids, essential oils, lubricants, lipids, skin coolants, vitamins, sunscreens, antioxidants, glycerine, catalysts, bleach particles, silicon dioxide particles, malodor reducing agents, odor-controlling materials, chelating agents, antistatic agents, softening agents, insect and moth repelling agents, colorants, bodying agents, drape and form control agents, smoothness agents, wrinkle control agents, sanitization agents, disinfecting agents, germ control agents, mold control agents, mildew control agents, antiviral agents, drying agents, stain resistance agents, soil release agents, fabric refreshing agents and freshness extending agents, chlorine bleach odor control agents, dye fixatives, dye transfer inhibitors, color maintenance agents, optical brighteners, color restoration/rejuvenation agents, anti-fading agents, whiteness enhancers, anti-abrasion agents, wear resistance agents, fabric integrity agents, anti-wear agents, anti-pilling agents, defoamers, anti-foaming agents, UV protection agents, sun fade inhibitors, anti-allergenic agents, enzymes, water proofing agents, fabric comfort agents, shrinkage resistance agents, stretch resistance agents, stretch recovery agents, skin care agents, synthetic or natural actives, antibacterial actives, antiperspirant actives, cationic polymers, dyes, and mixtures thereof.

The benefit agent in the core preferably comprises fragrance material (or simply “fragrance”), which may include one or more perfume raw materials. Fragrance is particularly suitable for encapsulation in the presently described delivery particles, as the fragrance-containing particles can provide freshness benefits across multiple touchpoints.

The term “perfume raw material” (or “PRM”) as used herein refers to compounds having a molecular weight of at least about 100 g/mol and which are useful in imparting an odor, fragrance, essence or scent, either alone or with other perfume raw materials. Typical PRMs comprise inter alia alcohols, ketones, aldehydes, esters, ethers, nitrites and alkenes, such as terpene. A listing of common PRMs can be found in various reference sources, for example, “Perfume and Flavor Chemicals”, Vols. I and II; Steffen Arctander Allured Pub. Co. (1994) and “Perfumes: Art, Science and Technology”, Miller, P. M. and Lamparsky, D., Blackie Academic and Professional (1994).

The PRMs may be characterized by their boiling points (B.P.) measured at the normal pressure (760 mm Hg), and their octanol/water partitioning coefficient (P), which may be described in terms of logP, determined according to the test method below. Based on these characteristics, the PRMs may be categorized as Quadrant I, Quadrant II, Quadrant III, or Quadrant IV perfumes, as described in more detail in U.S. Pat. No. 6,869,923. Suitable Quadrant I, II, III, and IV perfume raw materials are disclosed therein.

Perfume raw materials having a boiling point B.P. lower than about 250° C. and a logP lower than about 3 are known as Quadrant I perfume raw materials. Quadrant I perfume raw materials are preferably limited to less than 30% of the fragrance material.

The fragrance may comprise perfume raw materials that have a logP of from about 2.5 to about 4. It is understood that other perfume raw materials may also be present in the fragrance.

The core of the delivery particles of the present disclosure may comprise a partitioning modifier, which may facilitate more robust shell formation. The partitioning modifier may be combined with the core's perfume oil material prior to incorporation of the wall-forming monomers. The partitioning modifier may be present in the core at a level of from 0% to 95%, preferably from about 5% to about 55%, preferably from about 10% to about 50%, more preferably from about 20% to about 50%, even more preferably from about 25% to about 50%, by weight of the core.

The partitioning modifier may comprise a material selected from the group consisting of vegetable oil, modified vegetable oil, mono-, di-, and tri-esters of C₄-C₂₄ fatty acids, isopropyl myristate, dodecanophenone, lauryl laurate, methyl behenate, methyl laurate, methyl palmitate, methyl stearate, and mixtures thereof. The partitioning modifier may preferably comprise or even consist of isopropyl myristate. The modified vegetable oil may be esterified and/or brominated. The modified vegetable oil may preferably comprise castor oil and/or soy bean oil. US Patent Application Publication 20110268802, incorporated herein by reference, describes other partitioning modifiers that may be useful in the presently described delivery particles.

Optionally, the water phase may include an emulsifier. Non-limiting examples of emulsifiers include anionic surfactants (such as alkyl sulfates, alkyl ether sulfates, and/or alkyl benzenesulfonates), nonionic surfactants (such as alkoxylated alcohols, preferably comprising ethoxy groups), polyvinyl alcohol, and/or polyvinyl pyrrolidone. It may be that solubilized chitosan can provide emulsifying benefits in the present applications.

Emulsifier, if employed, is typically from about 0.1 to 40% by weight, preferably 0.2 to about 15% by weight, more typically 0.5 to 10% be weight, based on total weight of the aqueous phase.

The population of delivery particles may be provided as a slurry, preferably an aqueous slurry. The slurry can include one or more processing aids, which may include water, aggregate inhibiting materials such as divalent salts, or particle suspending polymers such as xanthan gum, guar gum, cellulose (preferably microfibrillated cellulose) and/or carboxy methyl cellulose.

The slurry can include one or more carriers selected from the group consisting of polar solvents, including but not limited to, water, ethylene glycol, propylene glycol, polyethylene glycol, glycerol; nonpolar solvents, including but not limited to, mineral oil, perfume raw materials, silicone oils, hydrocarbon paraffin oils; and mixtures thereof. Aqueous slurries may be preferred. The slurry may comprise non-encapsulated (of “free”) perfume raw materials that are different in identity and/or amount from those that are encapsulated in the cores of the delivery particles.

The slurry may include a deposition aid that may comprise a polymer selected from the group comprising: polysaccharides, such as chitosan, cationically modified starch, and/or cationically modified guar; polysiloxanes; poly diallyl dimethyl ammonium halides; copolymers of poly diallyl dimethyl ammonium chloride and polyvinyl pyrrolidone; a composition comprising polyethylene glycol and polyvinyl pyrrolidone; acrylamides; imidazoles; imidazolinium halides; polyvinyl amine; copolymers of poly vinyl amine and N-vinyl formamide; polyvinyl formamide, polyvinyl alcohol; polyvinyl alcohol crosslinked with boric acid; polyacrylic acid; polyglycerol ether silicone cross-polymers; polyacrylic acids, polyacrylates, copolymers of polyvinylamine and polvyinylalcohol oligomers of amines, in one aspect a diethylenetriamine, ethylene diamine, bis(3-aminopropyl)piperazine, N,N-Bis-(3-aminopropyl)methylamine, tris(2-aminoethyl)amine and mixtures thereof; polyethyleneimine, a derivatized polyethyleneimine, in one aspect an ethoxylated polyethyleneimine; a polymeric compound comprising, at least two moieties selected from the moieties consisting of a carboxylic acid moiety, an amine moiety, a hydroxyl moiety, and a nitrile moiety on a backbone of polybutadiene, polyisoprene, polybutadiene/styrene, polybutadiene/acrylonitrile, carboxyl-terminated polybutadiene/acrylonitrile or combinations thereof; pre-formed coacervates of anionic surfactants combined with cationic polymers; polyamines and mixtures thereof.

At least one population of delivery particles may be contained in an agglomerate and then combined with a distinct population of delivery particles and at least one adjunct material. Said agglomerate may comprise materials selected from the group consisting of silicas, citric acid, sodium carbonate, sodium sulfate, sodium chloride, and binders such as sodium silicates, modified celluloses, polyethylene glycols, polyacrylates, polyacrylic acids, zeolites and mixtures thereof.

Suitable equipment for use in the processes disclosed herein may include continuous stirred tank reactors, homogenizers, turbine agitators, recirculating pumps, paddle mixers, plough shear mixers, ribbon blenders, vertical axis granulators and drum mixers, both in batch and, where available, in continuous process configurations, spray dryers, and extruders. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany), Niro (Soeborg, Denmark), Hosokawa Bepex Corp. (Minneapolis, Minn., U.S.A.), Arde Barinco (New Jersey, U.S.A.).

### Adjunct Ingredient

The treatment compositions of the present disclosure may comprise one or more adjunct materials in addition to the delivery particles. The adjunct material may provide a benefit in the intended end-use of a composition, or it may be a processing and/or stability aid.

Suitable adjunct materials may include: surfactants, conditioning actives, deposition aids, rheology modifiers or structurants, bleach systems, stabilizers, builders, chelating agents, dye transfer inhibiting agents, dispersants, enzymes, and enzyme stabilizers, catalytic metal complexes, polymeric dispersing agents, clay and soil removal/anti-redeposition agents, brighteners, suds suppressors, silicones, hueing agents, aesthetic dyes, additional perfumes and perfume delivery systems, structure elasticizing agents, carriers, hydrotropes, processing aids, anti-agglomeration agents, coatings, formaldehyde scavengers, and/or pigments. Preferably, the adjunct materials comprise additional fabric conditioning agents, dyes, pH control agents, solvents, rheology modifiers, structurants, cationic polymers, surfactants, perfume, additional perfume delivery systems, chelants, antioxidants, preservatives, or mixtures thereof.

Depending on the intended form, formulation, and/or end-use, compositions of the present disclosure might not contain one or more of the following adjuncts materials: bleach activators, surfactants, builders, chelating agents, dye transfer inhibiting agents, dispersants, enzymes, and enzyme stabilizers, catalytic metal complexes, polymeric dispersing agents, clay and soil removal/anti-redeposition agents, brighteners, suds suppressors, dyes, additional perfumes and perfume delivery systems, structure elasticizing agents, fabric softeners, carriers, hydrotropes, processing aids, structurants, anti-agglomeration agents, coatings, formaldehyde scavengers, and/or pigments.

The precise nature of these additional components, and levels of incorporation thereof, will depend on the physical form of the composition and the nature of the operation for which it is to be used. However, when one or more adjuncts are present, such one or more adjuncts may be present as detailed below. The following is a non-limiting list of suitable additional adjuncts.

### A. Surfactants

The compositions of the present disclosure may comprise surfactant. Surfactants may be useful for providing, for example, cleaning benefits. The compositions may comprise a surfactant system, which may contain one or more surfactants.

The compositions of the present disclosure may include from about 0.1% to about 70%, or from about 2% to about 60%, or from about 5% to about 50%, by weight of the composition, of a surfactant system. Liquid compositions may include from about 5% to about 40%, by weight of the composition, of a surfactant system. Compact formulations, including compact liquids, gels, and/or compositions suitable for a unit dose form, may include from about 25% to about 70%, or from about 30% to about 50%, by weight of the composition, of a surfactant system.

The surfactant system may include anionic surfactant, nonionic surfactant, zwitterionic surfactant, cationic surfactant, amphoteric surfactant, or combinations thereof. The surfactant system may include linear alkyl benzene sulfonate, alkyl ethoxylated sulfate, alkyl sulfate, nonionic surfactant such as ethoxylated alcohol, amine oxide, or mixtures thereof. The surfactants may be, at least in part, derived from natural sources, such as natural feedstock alcohols.

Suitable anionic surfactants may include any conventional anionic surfactant. This may include a sulfate detersive surfactant, for e.g., alkoxylated and/or non-alkoxylated alkyl sulfate materials, and/or sulfonic detersive surfactants, e.g., alkyl benzene sulfonates. The anionic surfactants may be linear, branched, or combinations thereof. Preferred surfactants include linear alkyl benzene sulfonate (LAS), alkyl ethoxylated sulfate (AES), alkyl sulfates (AS), or mixtures thereof. Other suitable anionic surfactants include branched modified alkyl benzene sulfonates (MLAS), methyl ester sulfonates (MES), sodium lauryl sulfate (SLS), sodium lauryl ether sulfate (SLES), and/or alkyl ethoxylated carboxylates (AEC). The anionic surfactants may be present in acid form, salt form, or mixtures thereof. The anionic surfactants may be neutralized, in part or in whole, for example, by an alkali metal (e.g., sodium) or an amine (e.g., monoethanolamine). Due to the presence of cationic ester quat material, it may be desirable to limit the amount of anionic surfactant so as to avoid undesirable interactions of the materials; for example, the compositions may comprise less than 5%, preferably less than 3%, more preferably less than 1%, even more preferably less than 0.1%, by weight of the composition, of anionic surfactant.

The surfactant system may include nonionic surfactant. Suitable nonionic surfactants include alkoxylated fatty alcohols, such as ethoxylated fatty alcohols. Other suitable nonionic surfactants include alkoxylated alkyl phenols, alkyl phenol condensates, mid-chain branched alcohols, mid-chain branhed alkyl alkoxylates, alkylpolysaccharides (e.g., alkylpolyglycosides), polyhydroxy fatty acid amides, ether capped poly(oxyalkylated) alcohol surfactants, and mixtures thereof. The alkoxylate units may be ethyleneoxy units, propyleneoxy units, or mixtures thereof. The nonionic surfactants may be linear, branched (e.g., mid-chain branched), or a combination thereof. Specific nonionic surfactants may include alcohols having an average of from about 12 to about 16 carbons, and an average of from about 3 to about 9 ethoxy groups, such as C12-C14 EO7 nonionic surfactant.

Suitable zwitterionic surfactants may include any conventional zwitterionic surfactant, such as betaines, including alkyl dimethyl betaine and cocodimethyl amidopropyl betaine, C₈ to C₁₈ (for example from C₁₂ to C₁₈) amine oxides (e.g., C₁₂₋₁₄ dimethyl amine oxide), and/or sulfo and hydroxy betaines, such as N-alkyl-N,N-dimethylammino-1-propane sulfonate where the alkyl group can be C₈ to C₁₈, or from C₁₀ to C₁₄. The zwitterionic surfactant may include amine oxide.

Depending on the formulation and/or the intended end-use, the composition may be substantially free of certain surfactants. For example, liquid fabric enhancer compositions, such as fabric softeners, may be substantially free of anionic surfactant, as such surfactants may negatively interact with cationic ingredients.

### B. Conditioning Active

The compositions of the present disclosure may include a conditioning active. Compositions that contain conditioning actives may provide softness, anti-wrinkle, anti-static, conditioning, anti-stretch, color, and/or appearance benefits.

Conditioning actives may be present at a level of from about 1% to about 99%, by weight of the composition. The composition may include from about 1%, or from about 2%, or from about 3%, to about 99%, or to about 75%, or to about 50%, or to about 40%, or to about 35%, or to about 30%, or to about 25%, or to about 20%, or to about 15%, or to about 10%, by weight of the composition, of conditioning active. The composition may include from about 5% to about 30%, by weight of the composition, of conditioning active.

Conditioning actives suitable for compositions of the present disclosure may include quaternary ammonium ester compounds, silicones, non-ester quaternary ammonium compounds, amines, fatty esters, sucrose esters, silicones, dispersible polyolefins, polysaccharides, fatty acids, softening or conditioning oils, polymer latexes, or combinations thereof. Preferably the treatment composition is a fabric care composition where the one or more adjunct ingredients comprises quaternary ammonium ester material; such materials are particularly useful in fabric enhancing/conditioning/softening compositions.

The composition may include a quaternary ammonium ester compound, a silicone, or combinations thereof, preferably a combination. The combined total amount of quaternary ammonium ester compound and silicone may be from about 5% to about 70%, or from about 6% to about 50%, or from about 7% to about 40%, or from about 10% to about 30%, or from about 15% to about 25%, by weight of the composition. The composition may include a quaternary ammonium ester compound and silicone in a weight ratio of from about 1:10 to about 10:1, or from about 1:5 to about 5:1, or from about 1:3 to about 1:3, or from about 1:2 to about 2:1, or about 1:1.5 to about 1.5:1, or about 1:1.

The composition may contain mixtures of different types of conditioning actives. The compositions of the present disclosure may contain a certain conditioning active but be substantially free of others. For example, the composition may be free of quaternary ammonium ester compounds, silicones, or both. The composition may comprise quaternary ammonium ester compounds but be substantially free of silicone. The composition may comprise silicone but be substantially free of quaternary ammonium ester compounds.

### C. Deposition Aid

The compositions of the present disclosure may comprise a deposition aid. As described above, due to the synergistic benefits that flow from the ester quat material and the delivery particles of the present disclosure, relatively less (or even none) of a deposition aid may be require to provide comparable or even improved performance; alternatively, a deposition aid may be used in compositions of the present disclosure to boost performance even more.

Deposition aids can facilitate deposition of delivery particles, conditioning actives, perfumes, or combinations thereof, improving the performance benefits of the compositions and/or allowing for more efficient formulation of such benefit agents. The composition may comprise, by weight of the composition, from 0.0001% to 3%, preferably from 0.0005% to 2%, more preferably from 0.001% to 1%, or from about 0.01% to about 0.5%, or from about 0.05% to about 0.3%, of a deposition aid. The deposition aid may be a cationic or amphoteric polymer, preferably a cationic polymer.

Cationic polymers in general and their methods of manufacture are known in the literature. Suitable cationic polymers may include quaternary ammonium polymers known the “Polyquaternium” polymers, as designated by the International Nomenclature for Cosmetic Ingredients, such as Polyquaternium-6 (poly(diallyldimethylammonium chloride), Polyquaternium-7 (copolymer of acrylamide and diallyldimethylammonium chloride), Polyquaternium-10 (quaternized hydroxyethyl cellulose), Polyquaternium-22 (copolymer of acrylic acid and diallyldimethylammonium chloride), and the like.

The deposition aid may be selected from the group consisting of polyvinylformamide, partially hydroxylated polyvinylformamide, polyvinylamine, polyethylene imine, ethoxylated polyethylene imine, polyvinylalcohol, polyacrylates, and combinations thereof. The cationic polymer may comprise a cationic acrylate.

Deposition aids can be added concomitantly with delivery particles (at the same time with, e.g., encapsulated benefit agents) or directly/independently in the consumer product composition. The weight-average molecular weight of the polymer may be from 500 to 5000000 or from 1000 to 2000000 or from 2500 to 1500000 Dalton, as determined by size exclusion chromatography relative to polyethyleneoxide standards using Refractive Index (RI) detection. The weight-average molecular weight of the cationic polymer may be from 5000 to 37500 Dalton.

### D. Rheology Modifier/Structurant

The compositions of the present disclosure may contain a rheology modifier and/or a structurant. Rheology modifiers may be used to “thicken” or “thin” liquid compositions to a desired viscosity. Structurants may be used to facilitate phase stability and/or to suspend or inhibit aggregation of particles in liquid composition, such as the delivery particles as described herein.

Suitable rheology modifiers and/or structurants may include non-polymeric crystalline hydroxyl functional structurants (including those based on hydrogenated castor oil), polymeric structuring agents, cellulosic fibers (for example, microfibrillated cellulose, which may be derived from a bacterial, fungal, or plant origin, including from wood), di-amido gellants, or combinations thereof.

Polymeric structuring agents may be naturally derived or synthetic in origin. Naturally derived polymeric structurants may comprise hydroxyethyl cellulose, hydrophobically modified hydroxyethyl cellulose, carboxymethyl cellulose, polysaccharide derivatives and mixtures thereof. Polysaccharide derivatives may comprise pectine, alginate, arabinogalactan (gum Arabic), carrageenan, gellan gum, xanthan gum, guar gum and mixtures thereof. Synthetic polymeric structurants may comprise polycarboxylates, polyacrylates, hydrophobically modified ethoxylated urethanes, hydrophobically modified non-ionic polyols and mixtures thereof. Polycarboxylate polymers may comprise a polyacrylate, polymethacrylate or mixtures thereof. Polyacrylates may comprise a copolymer of unsaturated mono- or di-carbonic acid and C₁-C₃₀ alkyl ester of the (meth)acrylic acid. Such copolymers are available from Noveon inc under the tradename Carbopol Aqua 30. Cross-linked polymers, such as cross-linked polyacrylate and/or polymers and/or co-polymers, such as those that further include nonionic monomers such as acrylamide or methacrylamide monomers, may be useful as structurants. Another suitable structurant is sold under the tradename Rheovis CDE, available from BASF.

### E. Other Adjuncts

The treatment compositions of the present disclosure may contain other adjuncts that are suitable for inclusion in the product and/or for final usage. For example, the treatment compositions may comprise neat perfume, perfume delivery technologies (such as pro-perfumes and/or encapsulates having non-cross-linked-biopolymer wall materials), cationic surfactants, cationic polymers, solvents, suds supressors, or combinations thereof.

### Method of Making a Treatment Composition

The present disclosure further relates to methods for making a treatment composition, such as those treatment compositions and/or consumer product compositions described herein.

The method may comprise the steps of: providing a base composition, wherein the base composition comprises the treatment adjunct, and combining the population of delivery particles with the base composition. The population of delivery particles may preferably be provided as an aqueous slurry. The base composition is in the form of a liquid composition.

The delivery particles may be combined with the one or more adjunct ingredients when the delivery particles are in one or more forms, including a slurry form, neat particle form, and/or spray dried particle form, preferably slurry form. The delivery particles may be combined with such adjuncts by methods that include mixing and/or spraying.

The treatment compositions of the present disclosure can be formulated into any suitable form and prepared by any process chosen by the formulator. The one or more adjunct ingredients and the delivery particles may be combined in a batch process, in a circulation loop process, and/or by an in-line mixing process. Suitable equipment for use in the processes disclosed herein may include continuous stirred tank reactors, homogenizers, turbine agitators, recirculating pumps, paddle mixers, high shear mixers, static mixers, plough shear mixers, ribbon blenders, vertical axis granulators and drum mixers, both in batch and, where available, in continuous process configurations, spray dryers, and extruders.

The treatment composition may be placed into a container to form a consumer product, as described herein. The container may be a bottle, preferably a plastic bottle. The treatment composition may be placed into an aerosol or other spray container according to known methods.

### Method of Treatment

The present disclosure also relates to a method of treating a surface, preferably a fabric. In general, the method includes the step of contacting a surface, preferably a fabric, with a treatment composition according to the present disclosure, where the treatment composition includes a population of delivery particles as described herein.

Additionally or alternatively, the method may include the step of contacting a surface, preferably a fabric, with a population of delivery particles as described herein. The population of delivery particles may be contained in a treatment composition according to the present disclosure, preferably a fabric care composition.

The method may include the step of contacting a fabric, such as a garment, with a treatment composition. The treatment composition comprises a population of delivery particles. The contacting step results in one or more of the delivery particles being deposited on a surface of the fabric. The delivery particles comprise a core and a shell surrounding the core, where the core comprises a benefit agent, preferably a fragrance material that comprises one or more perfume raw materials. The shell comprises a polymeric material that is, for example, the reaction product of chitosan of a particular molecular weight and a cross-linking agent. Suitable treatment compositions and delivery particles are described in more detail above.

The contacting step may occur during a manual laundry process, for example in a wash basin as fabrics are treated by hand, or an automatic laundry process, for example in an automatic washing machine. The contacting step may occur during the wash cycle of an automatic washing machine; in such cases, the treatment composition may be a laundry detergent or a laundry additive. The contacting step may preferably occur during the rinse cycle of an automatic washing machine; in such cases, the treatment composition may be a fabric enhancer, preferably a liquid fabric enhancer. The contacting step may even occur during a drying step of a laundry process, for example in an automatic dryer machine; in such cases, the treatment composition may be in the form of a non-woven dryer sheet or a dryer bar. The contacting step may occur as a result of the treatment composition being directly applied to the fabric, for example in a pretreatment operation or in a “refreshing” step (e.g., for a fabric that has been used or worn since the last wash); in such cases, the treatment composition may be in the form of a liquid, a stick, or a spray, preferably a spray. Contacting the target fabrics relatively late in a laundering process, e.g., during a rinse cycle, improves the likelihood or efficiency of deposition onto the fabrics as they are less likely to be washed down the drain.

The contacting step may occur in the presence of water. The treatment composition may be diluted with water to form a treatment liquor. The treatment composition may be diluted from about 100-fold to about 1500-fold, preferably from 300-fold to about 1000-fold.

Liquors that comprise the disclosed compositions may have a pH of from about 3 to about 11.5. When diluted, such compositions are typically employed at concentrations of from about 500 ppm to about 15,000 ppm in solution. When the wash solvent is water, the water temperature typically ranges from about 5° C. to about 90° C. and, the water to fabric ratio may be typically from about 1:1 to about 30:1.

The dilution may occur in the drum of an automatic washing machine. The treatment composition may be placed into a dispensing drawer of an automatic washing machine. The treatment composition may be dispensed from the dispensing drawer to the drum during a treatment process.

The present disclosure also relates to a method of treating a fabric in an automatic washing machine. Typical treatment methods in such machines include a wash cycle, which typically include relatively higher shear agitation, and one or more rinse cycles, which typically include relatively lower shear agitation. In such cases, it may be preferred to treat the fabrics with delivery particles that are designed to sufficiently deliver the benefit agent under such conditions. For example, the method may include contacting fabrics in a wash cycle with a population of delivery particles that are characterized by a Volume-Weighted Ductile Energy of from about 4.5 to about 6.0, preferably from about 5.0 to about 5.5. The method may include contacting fabric in a rinse cycle with a population of delivery particles that are characterized by a Volume-Weighted Ductile Energy of from about 3.5 to about 5, preferably from about 3.8 to about 4.8, more preferably from about 4.0 to about 4.5.

The method may include contacting fabrics with a first population of delivery particles in the wash cycle and a second population of delivery particles in the rinse cycle, wherein the Volume-Weighted Ductile Energy of the first population is relatively greater than the Volume-Weighted Ductile Energy of the second population. The Volume-Weighted Ductile Energy of the first population is preferably from about 4.5 to about 6.0, preferably from about 5.0 to about 5.5, and the Volume-Weighted Ductile Energy of the second population is preferably from about 3.5 to about 5, preferably from about 3.8 to about 4.8, more preferably from about 4.0 to about 4.5.

As alluded to above, the method may further comprise a step of drying the fabric that has the one or more delivery particles on the surface of the fabric. The drying step may comprise a passive drying process, such as on a clothesline or drying rack. The drying step may comprise an automatic drying process, such as in an automatic dryer machine.

### COMBINATIONS

Specifically contemplated combinations of the disclosure are herein described in the following lettered paragraphs. These combinations are intended to be illustrative in nature and are not intended to be limiting.

A. A treatment composition comprising: a treatment adjunct, and a population of delivery particles, wherein the delivery particles comprise a core and a shell surrounding the core, wherein the core comprises a benefit agent, wherein the shell comprises a polymeric material, wherein the polymeric material comprises the reaction product of a biopolymer and a cross-linking agent, wherein the population of delivery particles is characterized by at least one, preferably at least two, of the following: (a) a Volume-Weighted Ductile Energy greater than about 3.5, based on fifty delivery particles selected at random being compressed by a blunt probe moving at 2 μm/s; (b) at least about 30%, by number, of the delivery particles are characterized as Completely Ductile particles, based on fifty delivery particles selected at random being compressed by a blunt probe moving at 2 μm/s; (c) less than 35%, by number, of the delivery particles are characterized as Single Rupture particles, based on fifty delivery particles selected at random being compressed by a blunt probe moving at 2 μm/s.

B. The treatment composition according to paragraph A, wherein the population of delivery particles is characterized by a Volume-Weighted Ductile Energy of from about 3.5 to about 10.0, preferably from about 3.5 to about 7.5, more preferably from about 3.8 to about 6.0, more preferably from about 4.0 to about 5.5, even more preferably from about 4.5 to about 5.2, based on fifty delivery particles selected at random being compressed by a blunt probe moving at 2 μm/s.

C. The treatment composition according to any of paragraphs A or B, wherein the population of delivery particles is characterized by having at least about 50%, by number, of the delivery particles being characterized as Completely Ductile particles, based on fifty delivery particles selected at random being compressed by a blunt probe moving at 2 μm/s.

D. The treatment composition according to any of paragraphs A-C, wherein the population of delivery particles is characterized by having less than 25%, by number, of the delivery particles being characterized as Single Rupture particles, based on fifty delivery particles selected at random being compressed by a blunt probe moving at 2 μm/s.

E. The treatment composition according to any of paragraphs A-D, wherein the biopolymer is selected from the group consisting of a polysaccharide, a protein, a nucleic acid, a polyphenolic compound, derivatives thereof, and combinations thereof.

F. The treatment composition according to any of paragraphs A-E, wherein the biopolymer is selected from the group consisting of chitosan, starch, modified starch, dextran, maltodextrin, dextrin, cellulose, modified cellulose, hemicellulose, chitin, alginate, lignin, gum, pectin, fructan, carrageenan, agar, pullulan, suberin, cutin, cutan, melanin, silk fibroin, gelatin, collagen, casein, sericin, fibroin, whey protein, zein, soy protein, plant storage protein, gluten, peptide, actin, polynucleotides, RNA, DNA, tannins, lignans, derivatives thereof, and combinations thereof.

G. The treatment composition according to any of paragraphs A-F, wherein the biopolymer is chitosan, a derivative thereof, or a combination thereof, preferably wherein the biopolymer is acid-treated chitosan, redox-initiator-treated chitosan, a derivative thereof, or a combination thereof.

H. The treatment composition according to any of paragraphs A-G, wherein the biopolymer is characterized by a molecular weight of from about 1 kDal to about 1000 kDal, preferably from about 50 kDal to about 600 kDal, more preferably from about 100 kDal to about 500 kDal, even more preferably from about 100 kDal to about 300 kDal, even more preferably from about 100 kDal to about 200 kDal.

I. The treatment composition according to any of paragraphs A-H, wherein the cross-linking agent is a material selected from the group consisting of a polyisocyanate, a polyacrylate, a poly(meth)acrylate, a polyisothiocyanate, an aldehyde, an epoxy compound, a polyphenol, a carbonyl halide, an aziridine, and combinations thereof, preferably a polyisocyanate, an epoxy compound, a bifunctional aldehyde, and combinations thereof, more preferably a polyisocyanate, even more preferably a polyisocyanate selected from the group consisting of: a polyisocyanurate of toluene diisocyanate; a trimethylol propane adduct of toluene diisocyanate; a trimethylol propane adduct of xylylene diisocyanate; 2,2′-methylenediphenyl diisocyanate; 4,4′-methylenediphenyl diisocyanate; 2,4′-methylenediphenyl diisocyanate;

[diisocyanato(phenyl)methyl]benzene; toluene diisocyanate; tetramethylxylidene diisocyanate; naphthalene-1,5-diisocyanate; 1,4-phenylene diisocyanate; 1,3-diisocyanatobenzene; derivatives thereof (such as pre-polymers, oligomers, and/or polymers thereof); and combinations thereof.

J. The treatment composition according to any of paragraphs A-I, wherein the reaction product is formed in a reaction in which the biopolymer is initially present in an aqueous phase and the cross-linking agent is initially present in an oil phase, wherein the cross-linking agent is present in the oil phase at a level of from about 1% to about 20%, preferably from about 2% to about 10%, more preferably from about 2.5% to about 5%, by weight of the oil phase, preferably wherein the biopolymer and the cross-linking agent are present in the reaction in a weight ratio of from about 1:10 to about 1:0.1.

K. The treatment composition according to any of paragraphs A-J, wherein the benefit agent is a fragrance material.

L. The treatment composition according to any of paragraphs A-K, wherein the core further comprises a partitioning modifier, preferably present at a level of from about 10% to about 50%, more preferably from about 20% to about 50%, even more preferably from about 30% to about 50%, by weight of the core, preferably wherein the partitioning modifier is selected from the group consisting of vegetable oil, modified vegetable oil, mono-, di-, and tri-esters of C₄-C₂₄ fatty acids, isopropyl myristate, dodecanophenone, lauryl laurate, methyl behenate, methyl laurate, methyl palmitate, methyl stearate, and mixtures thereof, more preferably isopropyl myristate.

M. The treatment composition according to any of paragraphs A-L, wherein the population of delivery particles are made by a process that includes at least one milling step, wherein the at least one milling step occurs at a temperature of at least about 15° C., preferably at least about 20° C., more preferably at least about 25° C., even more preferably from about 25° C. to about 35° C.

N. The treatment composition according to any of paragraphs A-M, wherein the delivery particles are characterized by a volume-weighted median particle size from about 1 to about 100 microns, preferably from about 10 to about 100 microns, preferably from about 15 to about 50 microns, more preferably from about 20 to about 40 microns, even more preferably from about 25 to about 35 microns.

O. The treatment composition according to any of paragraphs A-N, wherein the shells of the delivery particles degrade at least 60% in 60 days when tested according to test method OECD 301B.

P. The treatment composition according to any of paragraphs A-O, wherein the treatment adjunct is selected from the group consisting of surfactants, conditioning actives, deposition aids, rheology modifiers or structurants, bleach systems, stabilizers, builders, chelating agents, dye transfer inhibiting agents, dispersants, enzymes, enzyme stabilizers, catalytic metal complexes, polymeric dispersing agents, clay and soil removal/anti-redeposition agents, brighteners, suds suppressors, silicones, hueing agents, aesthetic dyes, neat perfume, additional perfume delivery systems, structure elasticizing agents, carriers, hydrotropes, processing aids, anti-agglomeration agents, coatings, formaldehyde scavengers, pigments, and mixtures thereof.

Q. The treatment composition according to any of paragraphs A-P, wherein the treatment adjunct comprises anionic surfactant, a cationic conditioning agent, or a mixture thereof.

R. The treatment composition according to any of paragraphs A-Q, wherein the treatment composition is a fabric care composition, a hard surface cleaner composition, a dish care composition, a hair care composition, a body cleansing composition, or a mixture thereof, preferably a fabric care composition, more preferably a fabric care composition that is a laundry detergent composition, a fabric conditioning composition, a laundry additive, a fabric pre-treat composition, a fabric refresher composition, or a mixture thereof.

S. The treatment composition according to any of paragraphs A-R, wherein the treatment composition is in the form of a liquid composition, a granular composition, a hydrocolloid, a single-compartment pouch, a multi-compartment pouch, a dissolvable sheet, a pastille or bead, a fibrous article, a tablet, a stick, a bar, a flake, a foam/mousse, a non-woven sheet, or a mixture thereof, preferably a liquid composition.

T. The treatment composition according to any of paragraphs A-S, wherein the treatment composition comprises from about 50% to about 99%, by weight of the treatment composition, of water, preferably from about 60% to about 98%, more preferably from about 80% to about 96%, by weight of the treatment composition, of water.

U. A method of making a treatment composition according to any of paragraphs A-T, the method comprising the steps of: providing a base composition, wherein the base composition comprises the treatment adjunct, and combining the population of delivery particles with the base composition.

V. The method according to paragraph U, wherein the population of delivery particles are provided as an aqueous slurry.

W. The method according to any of paragraphs U or V, wherein the base composition is in the form of a liquid composition.

X. A method of treating a surface, the method comprising the step of: contacting the surface, preferably a fabric, with a treatment composition according to any of paragraphs A-T.

Y. A method of treating a fabric in an automatic washing machine, the method comprising the steps of: contacting fabrics with a first population of delivery particles in a wash cycle and a second population of delivery particles in a rinse cycle, wherein the Volume-Weighted Ductile Energy of the first population is relatively greater than the Volume-Weighted Ductile Energy of the second population, preferably wherein the Volume-Weighted Ductile Energy of the first population is from about 4.5 to about 6.0, preferably from about 5.0 to about 5.5, and preferably wherein the Volume-Weighted Ductile Energy of the second population is preferably from about 3.5 to about 5, preferably from about 3.8 to about 4.8, more preferably from about 4.0 to about 4.5.

### TEST METHODS

It is understood that the test methods disclosed in the Test Methods section of the present application should be used to determine the respective values of the parameters of Applicant's claimed subject matter as claimed and described herein.

### Mechanical Properties of Delivery Particles

The mechanical properties of the core:shell delivery particles as described herein are determined according to the following methods.

The general technique is a well-known methodology already defined in the literatures in Zhang, Z., Saunders, R. and Thomas, C. R., Micromanipulation measurements of the bursting strength of single microcapsules, Journal of Microencapsulation 16(1), 117-124 (1999). Based on the measurements obtained via the methodology, a number of useful characteristics can be observed and described, as provided in more detail below.

### 1. Extraction of Particles

When a population of delivery particles is provided as part of a finished product composition, the particles will need to be extracted in order to perform the analysis described herein.

Except where otherwise specified herein, the preferred method to isolate delivery particles from finished products is based on the fact that the density of most such delivery particles is different from that of water. The finished product is mixed with water in order to dilute and/or release the delivery particles. The diluted product suspension is centrifuged to speed up the separation of the delivery particles. Such delivery particles tend to float or sink in the diluted solution/dispersion of the finished product. Using a pipette or spatula, the top and bottom layers of this suspension are removed and undergo further rounds of dilution and centrifugation to separate, clean, and/or enrich the delivery particles. The delivery particles are observed using an optical microscope equipped with crossed-polarized filters or differential interference contrast (DIC), for example at total magnifications of 10× and 40×. The microscopic observations provide an initial indication of the presence, size, quality and aggregation of the delivery particles.

For extraction of delivery particles from a liquid fabric enhancer finished product conduct the following procedure:
1. Place three aliquots of approximately 20 ml of liquid fabric enhancer into three separate 50 ml centrifuge tubes and dilute each aliquot 1:1 with DI water (e.g. 20 ml fabric enhancer+20 ml DI water), mix each aliquot well and centrifuge each aliquot for 30 minutes at approximately 10000×g.
2. After centrifuging per Step 1, discard the bottom water layer (around 10 ml) in each 50 ml centrifuge tube then add 10 ml of DI water to each 50 ml centrifuge tube.
3. For each aliquot, repeat the process of centrifuging, removing the bottom water layer and then adding 10 ml of DI water to each 50 ml centrifuge tube two additional times.
4. Remove the top layer with a spatula or a pipette, and
5. Transfer this top layer into a 1.8 ml centrifuge tube and centrifuge for 5 minutes at approximately 20000×g.
6. Remove the top layer with a spatula and transfer into a new 1.8 ml centrifuge tube and add DI water until the tube is completely filled, then centrifuge for 5 minutes at approximately 20000×g.
7. Remove the bottom layer with a fine pipette and add DI water until tube is completely filled and centrifuge for 5 minutes at approximately 20000×g.
8. Repeat step 7 for an additional 5 times (6 times in total).

If both a top layer and a bottom layer of enriched delivery particles appear in the above described step 1, then, immediately move to step 3 (i.e., omit step 2) and proceed steps with steps 4 through 8. Once those steps have been completed, also remove the bottom layer from the 50 ml centrifuge tube from step 1, using a spatula or/and a pipette. Transfer the bottom layer into a 1.8 ml centrifuge tube and centrifuge 5 min at approximately 20000×g. Remove the bottom layer in a new tube and add DI water until the tube is completely filled then centrifuge for 5 minutes approximately 20000×g. Remove the top layer (water) and add DI water again until the tube is full. Repeat this another 5 times (6 times in total). Recombine the delivery particle enriched and isolated top and bottom layers back together.

If the fabric enhancer has a white color or is difficult to distinguish the delivery particle enriched layers add 4 drops of dye (such as Liquitint Blue JH 5% premix from Milliken & Company, Spartanburg, South Carolina, USA) into the centrifuge tube of step 1 and proceed with the isolation as described.

For extraction of delivery particles from solid finished products that disperse readily in water, mix 1 L of DI water with 20 g of the finished product (e.g. detergent foams, films, gels and granules; or water-soluble polymers; soap flakes and soap bars; and other readily water-soluble matrices such as salts, sugars, clays, and starches). When extracting delivery particles from finished products which do not disperse readily in water, such as waxes, dryer sheets, dryer bars, and greasy materials, it may be necessary to add detergents, agitation, and/or gently heat the product and diluent in order to release the delivery particles from the matrix. The use of organic solvents or drying out of the delivery particles should be avoided during the extraction steps as these actions may damage the delivery particles during this phase.

For extraction of delivery particles from liquid finished products which are not fabric softeners or fabric enhancers (e.g., liquid laundry detergents, liquid dish washing detergents, liquid hand soaps, lotions, shampoos, conditioners, and hair dyes), mix 20 ml of finished product with 20 ml of DI water. If necessary, NaCl (e.g., 1 to 4 g NaCl) can be added to the diluted suspension in order to increase the density of the solution and facilitate the delivery particles floating to the top layer. If the product has a white color which makes it difficult to distinguish the layers of delivery particles formed during centrifugation, a water-soluble dye can be added to the diluent to provide visual contrast.

The water and product mixture is subjected to sequential rounds of centrifugation, involving removal of the top and bottom layers, re-suspension of those layers in new diluent, followed by further centrifugation, isolation and re-suspension. Each round of centrifugation occurs in tubes of 1.5 to 50 ml in volume, using centrifugal forces of up to 20,000×g, for periods of 5 to 30 minutes. At least six rounds of centrifugation are typically needed to extract and clean sufficient delivery particles for testing. For example, the initial round of centrifugation may be conducted in 50 ml tubes spun at 10,000×g for 30 mins, followed by five more rounds of centrifugation where the material from the top and bottom layers is resuspended separately in fresh diluent in 1.8 ml tubes and spun at 20,000×g for 5 mins per round.

If delivery particles are observed microscopically in both the top and bottom layers, then the delivery particles from these two layers are recombined after the final centrifugation step, to create a single sample containing all the delivery particles extracted from that product. The extracted delivery particles should be analyzed as soon as possible but may be stored as a suspension in DI water for up to 14 days before they are analyzed.

One skilled in the art will recognize that various other protocols may be constructed for the extraction and isolation of delivery particles from finished products and will recognize that such methods require validation via a comparison of the resulting measured values, as measured before and after the delivery particles' addition to and extraction from finished product.

### 1. Preparation of Particles for Measurement of Mechanical Properties

10 μl of the population of capsules, typically in slurry form, is diluted in 1.5 ml of distilled water. To ensure the sample is homogenous, the sample is mixed for several seconds. Once mixed/homogenous, 5 μl of the diluted population of capsules is spread on the glass microslide of the Nanoindenter.

### 3. Methodology for Measurement of Mechanical Properties

The measurements are performed using an iNanoR Nanoindenter (available from KLA, United States), equipped with a flat-end probe with Poisson's Ratio 0.07, Modulus 1140 GPa and diameter 100 μm. The Frame Stiffness is equal to 8.8*10⁵ N/m. The probe speed is set to 2 μm/s.

For a given population of delivery particles, 50 capsules are selected from the population at random, and various characteristics of each capsule are measured, as described in more detail below. In short, the diameter of each particle is measured, and then each particle is compressed with the probe of the iNanoR Nanoindenter. Measurements such as the Speed of compression, the Depth of compression, and the related Load are recorded.

Based on the measurements, the diameter and speed of rupture of individual capsules can be determined, as described below.

### A. Determination of Individual Particle Diameter

For the purposes of the measurements described in this section, the diameter of an individual delivery particle is defined as the height of the capsule, measured along on the vertical axis perpendicular to the microslide or substrate upon which the delivery particle population is placed.

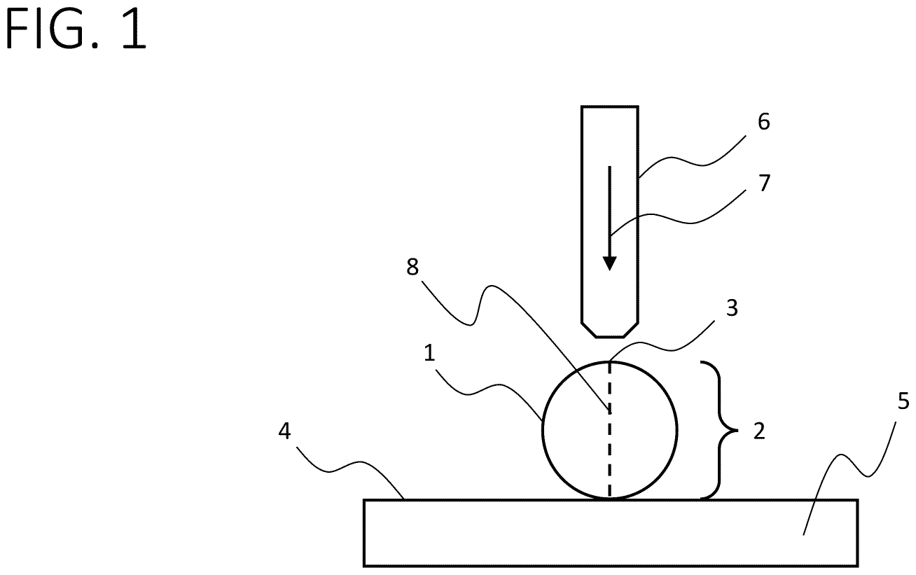

FIG. 1 shows the basic set-up for measuring the diameter of a delivery particle (or capsule, as used herein). The diameter of each delivery particle 1 is calculated by determining the height 2 of the top surface 3 of the delivery particle 1 relative to the surface 4 of the substrate 5 upon which the delivery particle 1 is placed, using the probe 6 of the iNanoR Nanoindenter. The arrow shows the direction of compression 7. The dashed line shows the vertical axis 8 of the delivery particle 1. The height 2 of the surface 4 of the substrate 5 is measured at a position of the microslide 150 um right and 150 um down relative from the centre of the delivery particle 1. Typically, the diameter is reported in microns.

The diameter may be calculated by the following equation.

Diameter=Height of top Capsule Surface−Height of Substrate

### B. Determination of Speed of Rupture

For each delivery particle, the speed of rupture, if any, is determined. Based on the speed of rupture, if any, measured during the capsule compression test, the particle is assigned to one of three categories, as described in more detail below.

As described above, the speed of compression of the probe is set to 2 μm/s for each capsule of the measurement. As the capsule is compressed, the speed, depth, and load of the probe is recorded.

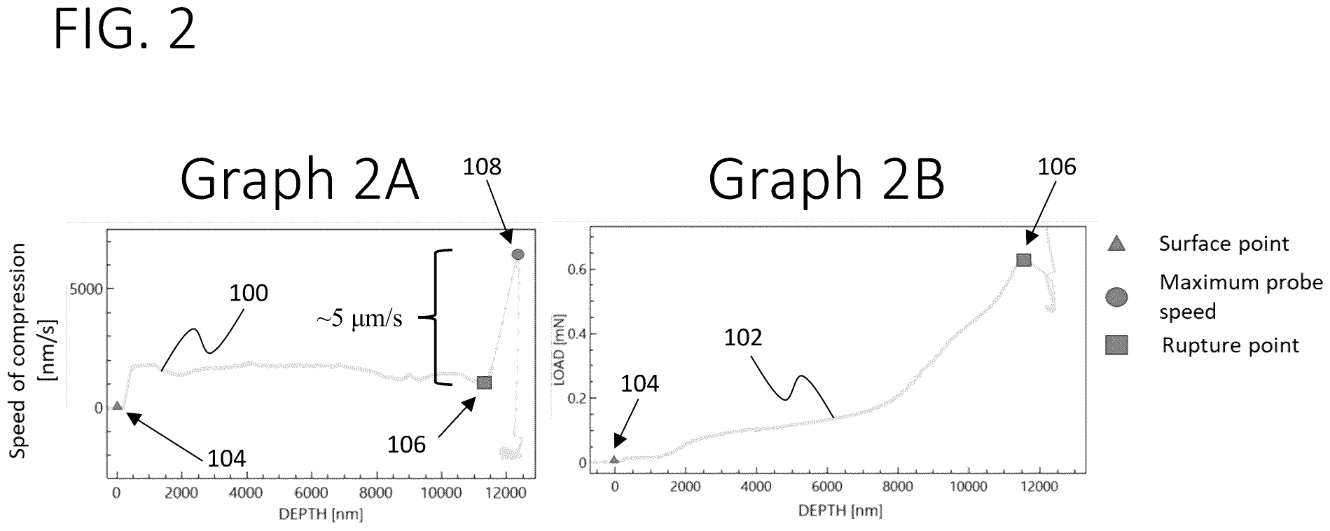

From the measurements, a Speed-Depth curve and a Load-Depth curve can be created. FIG. 2 shows such curves for an exemplary particle that ruptures. Graph 2A shows the Speed-Depth Curve 100, while Graph 2B shows how the load (measured in mN) varies throughout the measurement in function of the Depth, presented as a Load-Depth curve 102. For each, the Depth corresponds to the amount of travel of the probe throughout the compression.

As shown in Graph 2A, the speed of compression can vary throughout the measurement of a magnitude around 50 nm/s due to variations in the mechanical resistance exercised by the capsule onto the probe. At the point of zero Depth, the probe comes into contact with the capsule; this point on the curve is identified by the triangle 104.

As shown in Graph 2B, for the illustrative capsule, the Load increases until the rupture point of the capsule, which is characterized by the Maximum Load; this point on the curve is identified by the square in Graph 2B. The parallel point on the Speed-Depth curve of Graph 2A is also identified with a square (“rupture point”).

After the rupture point, the compression speed suddenly increases because the capsule is no longer exercising significant resistance to the probe compression. After the point of rupture of the capsule, the point of Maximum probe speed can be identified; this point on the curve is represented by the circle 108 in Graph 2A.

As used herein, the Speed of Rupture is defined as the difference in speed between the maximum of the speed of the probe (after the rupture) and the probe speed at the point of rupture (which correlates to the probe Depth at the point at which the Maximum load is observed). The formula to calculate the Speed of Rupture is reported in Equation (1.1).

Speed of Rupture=Maximum probe speed−probe speed at Rupture point (1.1)

For the illustrative capsule in Graph 2A, it can be seen that the speed of rupture in view of the maximum probe speed (represented by the circle) has a value of around 5 μm/s.

### 4. Determination of Volume Weighted Ductile Energy and Proportions of Ductile Capsules

To determine the Volume-weighted Ductile Energy for the population of delivery particles, 50 capsules are selected from the population at random, and various characteristics of each capsule are measured, as described in more detail below. Based on the Speed of Rupture (if any), each individual capsule is assigned to one of three categories (Ductile, Single Rupture, or Multiple Rupture), as described below. Using area under the Load vs. Depth curves, the measured Ductile Energy of the individual capsules is determined. From these measurements, the volume-weighted Ductile Energy of the delivery particle population is determined. Further, the relative proportions of particles that are ductile or that exhibit “single rupture” behavior can be determined from the data.

### A. Categorizing the Individual Capsules

Each individual capsule is categorized in the terms of their behaviour in below three categories based on the speed of rupture and number of maxima peaks on the Load-Depth curves: Completely Ductile behaviour; Single Rupture behaviour; or Multiple rupture behaviour.

i. Definition of a Completely Ductile Capsule

“Completely Ductile” capsules, as defined herein, are characterized by a difference between the Maximum probe speed minus the standard probe speed that does not exceed 200 nm when compressed by a blunt probe moving at 2 μm/s, thus no speed of rupture can be defined.

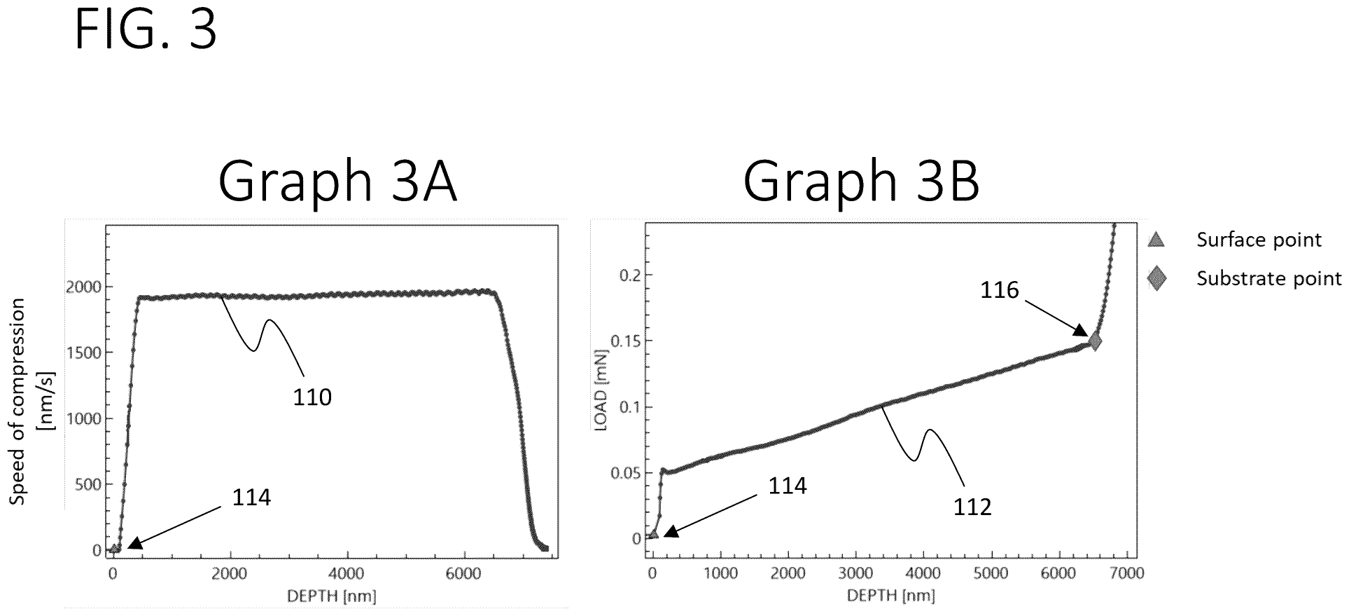

FIG. 3 shows compression curves for a Completely Ductile particle. As it can be seen in the Speed-Depth curve 110 of Graph 3A, the speed of compression throughout the measurement does not exceed the standard compression speed of 2 μm/s. Additionally, no drop or peaked maximum of Load is observed in the Load-Depth curve 112 of Graph 3B.

Based on the curves 110, 112 of Graphs 3A and 3B, the particle does not show any point of rupture; for example, there is no relative increase in compression speed nor any drop in the Load that results in a maxima peak. Hence, the particle is defined as Completely Ductile.

At the point of zero Depth, the probe comes into contact with the particle; this point on the curves 110, 112 is identified by the triangle 114. During the last part of the measurement at a probe Depth around 6500 nm, it can be noted that the speed of compression approaches 0 nm/s as the whole diameter of the capsule was compressed and the tip is exercising pressure towards the substrate upon which the capsule is located. The diamond 116 in Graph 3B represents the point at which the probe touches the substrate, which is associated to an exponential increase in Load given that the substrate is a highly stiff material, such as a glass microslide.

ii. Definition of a Single-Rupture Capsule

A “single-rupture” capsule, as defined herein, is characterized by exhibiting a single capsule rupture point. A “single-rupture” capsule is characterized by a Speed of Rupture that exceeds 200 nm a single time when compressed by a blunt probe moving at 2 μm/s.

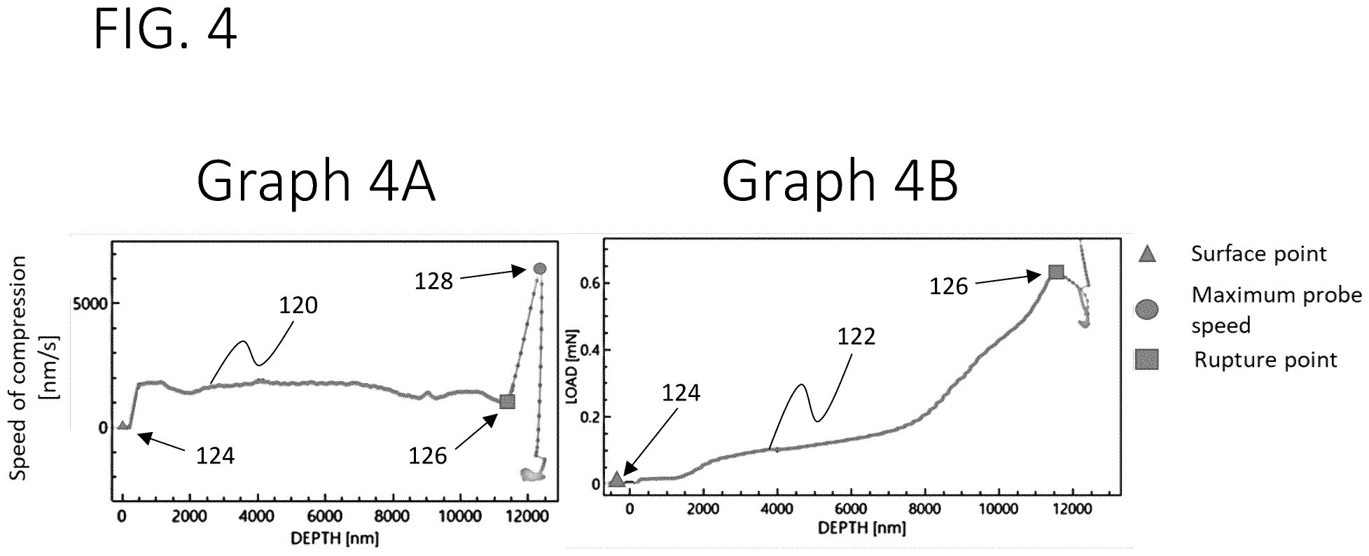

FIG. 4 shows compression curves for a Single-Rupture particle. At the point of zero Depth, the probe comes into contact with the particle; this point on the 120, 122 is identified by the triangle 124. In the Speed-Depth curve 120 of Graph 4A, it can be seen that the speed of compression exceeds 2 μm/s after the rupture point is detected; the rupture point is indicated by the squares 126 in Graphs 4A and 4B. The Maximum speed of the probe is then identified by the circle 128 in Graph 4A. As shown in Graph 4B, the Load-Depth curve 122 is characterized by a single maxima peak (shown at square 126), which suggests that the particle demonstrates single-rupture behavior.

iii. Definition of a Multiple-Rupture Capsule

A “multiple-rupture” capsule is characterized by exhibiting multiple capsule rupture points when compressed by a blunt probe moving at 2 μm/s. A “multiple-rupture” capsule is characterized by a Speed of Rupture that exceeds 200 nm multiple times when compressed by a blunt probe moving at 2 μm/s. Typically, a multiple-rupture capsules is characterized by two or three rupture points, although more are possible.

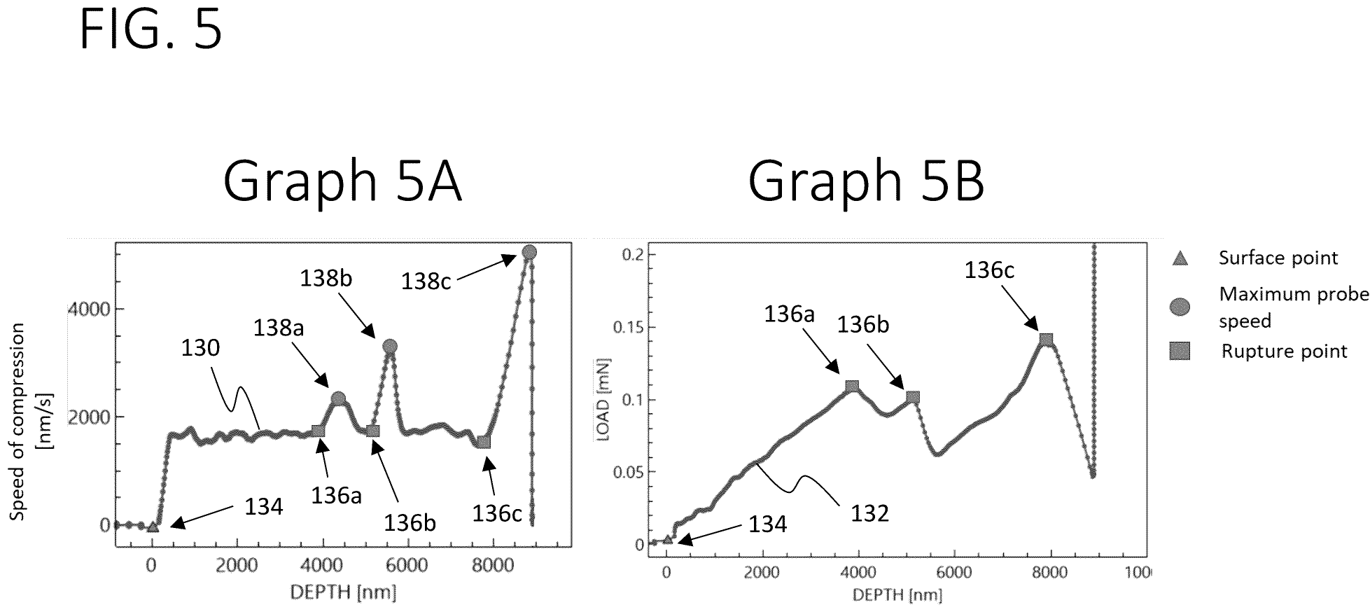

FIG. 5 shows compression curves for a Multiple-Rupture particle. At the point of zero Depth, the probe comes into contact with the particle; this point on the curves 130, 132 is identified by the triangle 134. As it can be seen in the Speed-Depth curve 130 Graph 5A and the Load-Depth curve 132 of Graph 5B, multiple rupture points are present. More precisely, the curves indicate three points of rupture associated with three maxima peaks of maximum load (squares 136a, 136b, 136c) and three relative increases in speed of compression; the Maximum probe speed is indicated by circles 136a, 136b, 136c.

### B. Determination of Category Proportions

Based on the number of capsules placed into each category, the population may be characterized by the relative amount of capsules in any given category.

For example, the population may be described by the proportion and/or percentage of capsules that are Completely Ductile (e.g., number of capsules categorized as being Completely Ductile, divided by the total number of capsules measured [i.e., 50]).

The population may be described by the proportion and/or percentage of capsules that exhibit Single-Rupture behavior (e.g., number of capsules categorized as being Single Rupture capsules, divided by the total number of capsules measured [i.e., 50]).

The population may be described by the proportion and/or percentage of capsules that exhibit Multiple-Rupture behavior (e.g., number of capsules categorized as being Multiple Rupture capsules, divided by the total number of capsules measured [i.e., 50]).

### C. Calculation of Ductile Energy for the Capsules, by Category

To calculate the Ductile Energy of the individually measured delivery particles in the population, the Area underneath certain points on the Load-Depth curve of the capsule is determined. The Area is calculated according to Equations 1.2-1.4 below and may be automatically calculated by any suitable program, for example by exporting the data points to MICROSOFT EXCEL® and using the program to determine the Area below the relevant points.

i. Ductile Energy of Completely Ductile Capsules

For Completely Ductile Capsules, the Ductile Energy is calculated as the Area underneath the Load vs Depth curve from the point the surface of the capsule is touched by the probe to the point where the substrate is touched by the probe.

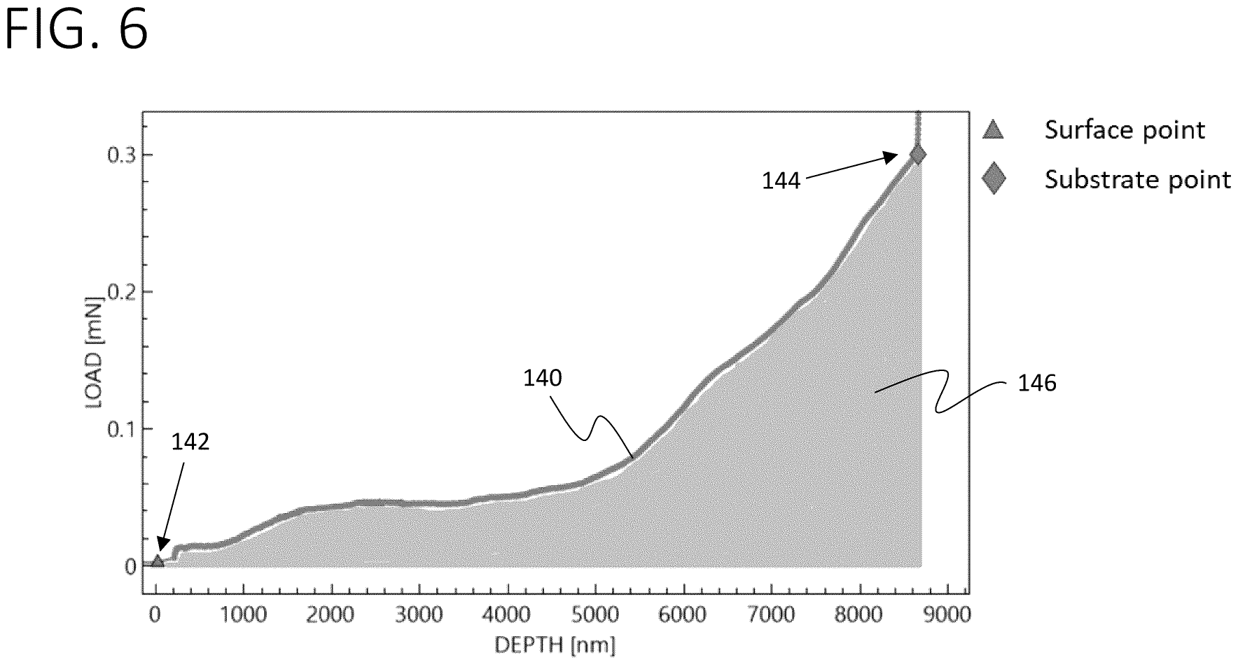

FIG. 6 shows a Load-Depth curve 140 for an illustrative Completely Ductile particle. The point the surface of the capsule is touched by the probe is represented in FIG. 6 as a triangle 142. The point where the substrate is touched by the probe is represented in FIG. 6 as a diamond 144. The Area underneath the relevant portion of the curve is represented by the shaded area 146 in FIG. 6.

The Area is calculated via the integral of the Load times the differential of the Depth over the interval ranging from the surface of the capsule to the substrate point, as shown in Equation (1.2).

$$Ductile Energy = {\int }_{{Depth}_{surf}}^{{Depth}_{s u b s t r a t e}} F \cdot d Depth \qquad ( 1.2 )$$

where Depth_{surf} is the Depth value at the moment when the capsule is touched by the probe (triangle 142 in FIG. 6), while Depth_{substrate} is the Depth value at the moment when the substrate is touched (diamond 144 in FIG. 6), while F is the Load measured by the probe during the compression.
ii. Ductile Energy of Single-Rupture Capsules

For Single-Rupture Capsules, the Ductile Energy is calculated as the Area underneath the Load vs Depth curve from the point the surface of the capsule is touched by the probe to the (first and only) rupture point of the capsule.

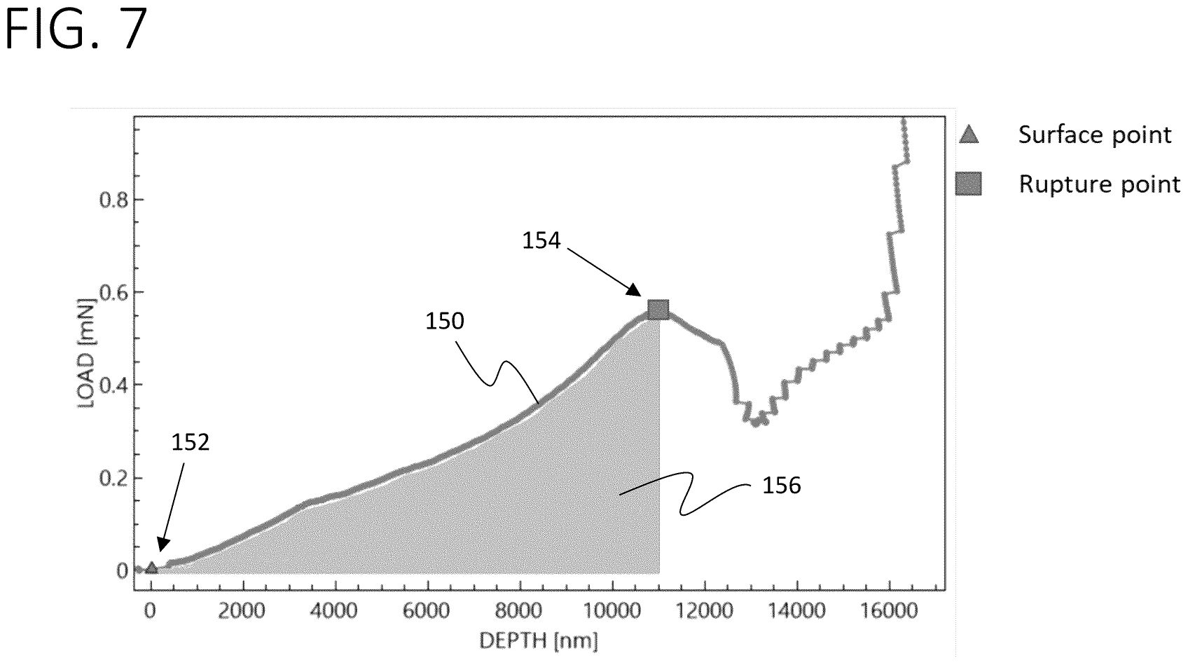

FIG. 7 shows a Load-Depth 150 curve for an illustrative Single-Rupture particle. The point the surface of the capsule is touched by the probe is represented in FIG. 7 as a triangle 152. The point at which the capsule ruptures is represented in FIG. 7 as a square 154. The Area underneath the relevant portion of the curve 150 is represented by the shaded area 156 in FIG. 7.

The Area is calculated via the integral of the Load times the differential of the Depth over the interval ranging from the surface of the capsule to the rupture point, as shown in Equation (1.3).

$$Ductile Energy = {\int }_{{Depth}_{surf}}^{{Depth}_{rup}} F \cdot d Depth \qquad ( 1.3 )$$

where Depth_{surf} is the Depth value at the moment when the capsule is touched by the probe (triangle 152 in FIG. 7), while Depthᵣᵤₚ is the Depth value at the point of rupture (square 154 in FIG. 7).
iii. Ductile Energy of Multiple-Rupture Capsules

For Multiple-Rupture Capsules, the Ductile Energy is calculated as the Area underneath the Load vs Depth curve from the point the surface of the capsule is touched by the probe to the to the last rupture point experienced by the capsule.

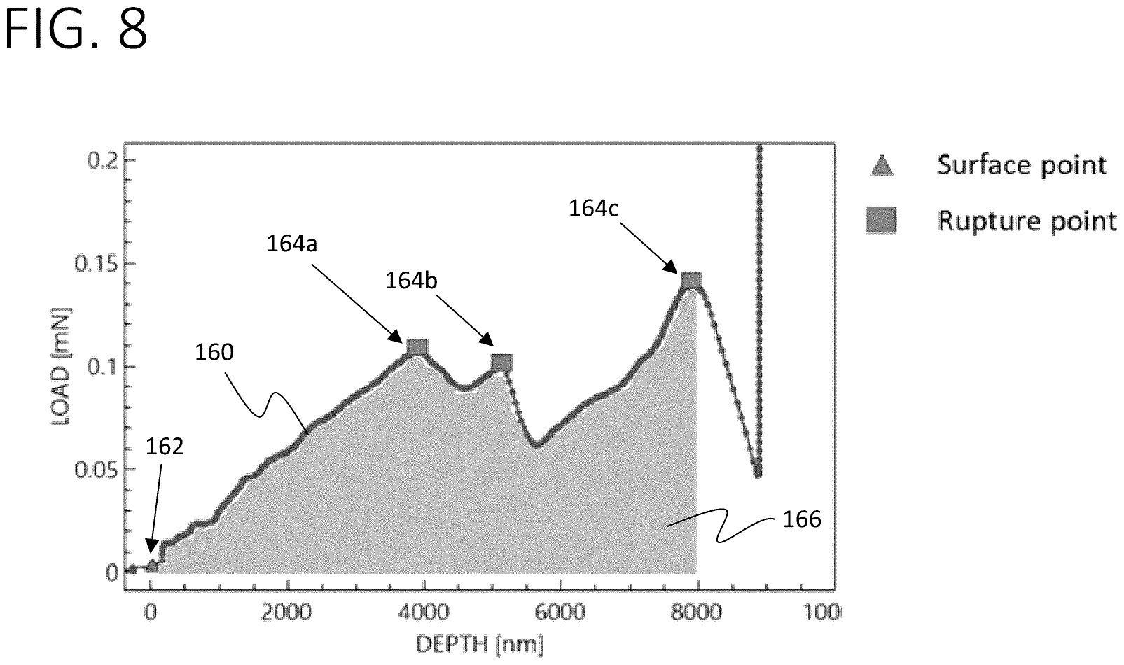

FIG. 8 shows a Load-Depth curve 160 for an illustrative Multiple-Rupture particle. The point the surface of the capsule is touched by the probe is represented in FIG. 8 as a triangle 162. The three points at which the capsule ruptures are represented in FIG. 8 as three squares 164a, 164b, 164c; the third and last rupture point 164c occurs at a probe Depth of approximately 8000 nm. The Area underneath the relevant portion of the curve 160 is represented by the shaded area 166 in FIG. 8.

The Area is calculated via the integral of the Load times the differential of the Depth over the interval ranging from the surface of the capsule to the last rupture point, as shown in Equation (1.4).

$$Ductile Energy = {\int }_{{disp}_{surf}}^{{disp}_{last rup}} F \cdot d Depth \qquad ( 1.4 )$$

where Disp_{surf} is the Depth value at the moment when the capsule is touched by the probe (triangle 162 in FIG. 8), while Disp_{last rupt} is the Depth value at the last rupture experienced by the capsule (the third/right-most square 164c in FIG. 8).

### D. Calculation of Volume-Weighted Ductile Energy

The Volume-Weighted Ductile Energy of the population of delivery particles is determined using the diameter and Ductile Energy values calculated for the individual particles, following a log transformation and a re-scaling of the Ductile Energy values.

i. Log Transformation

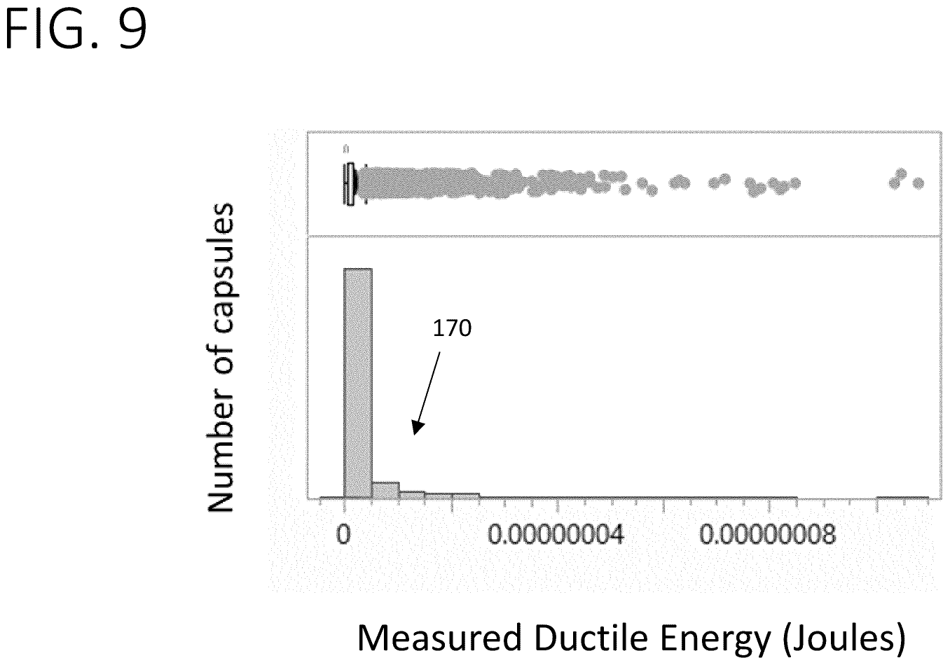

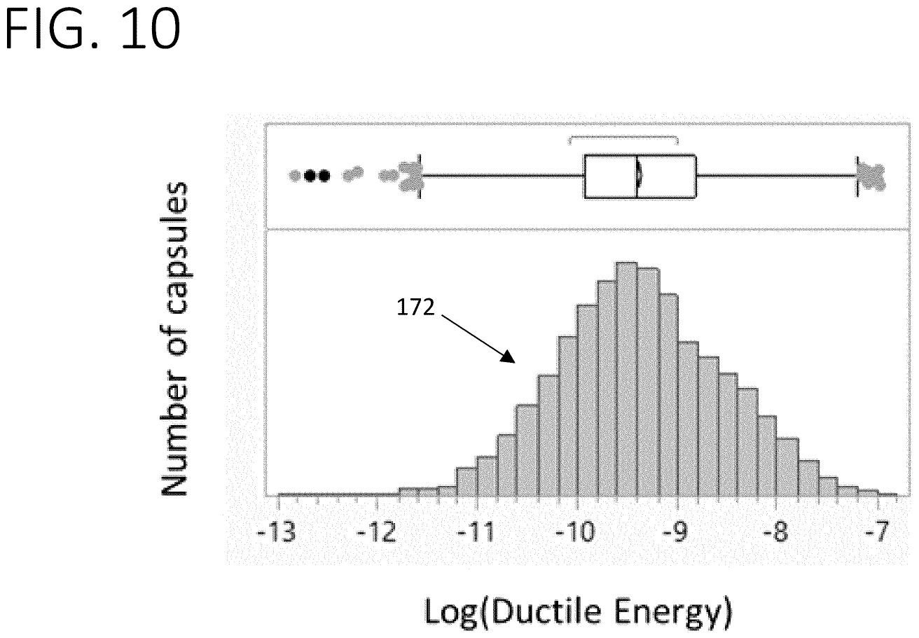

FIG. 9 shows a distribution 170 of the measured Ductile Energy values (in Joules) of an illustrative population of delivery particles. As seen in FIG. 9, the distribution of measured Ductile Energy data does not follow a normal distribution. In order to remove the skewedness of the data, a log transformation is carried out. The resulting distribution 172 of log (Ductile Energy) values is shown in FIG. 10.

ii. Rescaling

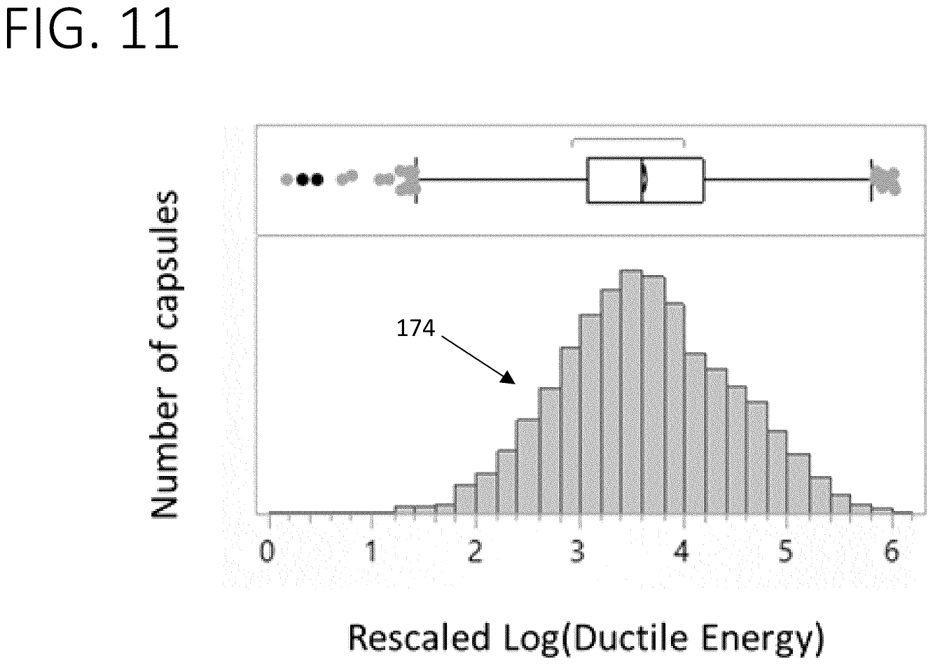

To ensure that the data points are non-negative for modeling purposes, the log (Ductile Energy) values are re-scaled and converted to Rescaled Log (Ductile Energy) values by adding a Ductile Energy Rescaling Factor to each data point, as shown in Equation 1.5. The Ductile Energy Rescaling Factor is equal to the negative log of the minimum measured Ductile Energy. Within the illustrative sample, this corresponds to 13, which is relative to the minimum measurable Ductile Energy of 10⁻¹³ J. The resulting distribution 174 of Rescaled Log (Ductile Energy) values is shown in FIG. 11.

Rescaled Log (Ductile Energy)=log (Ductile Energy)+13 (1.5)

iii. Prediction of Rescaled Log Ductile Energy by Diameter

For the whole population of capsules analyzed, the prediction of Rescaled Log (Ductile Energy) data as a function of the diameter is obtained in order to extrapolate the capsule the Volume Weighted Ductile Energy.

In this calculation, the Rescaled Log (Ductile Energy) data will be defined as response variable (y), while the diameter will be defined as regressor variable (x) for the following equations.

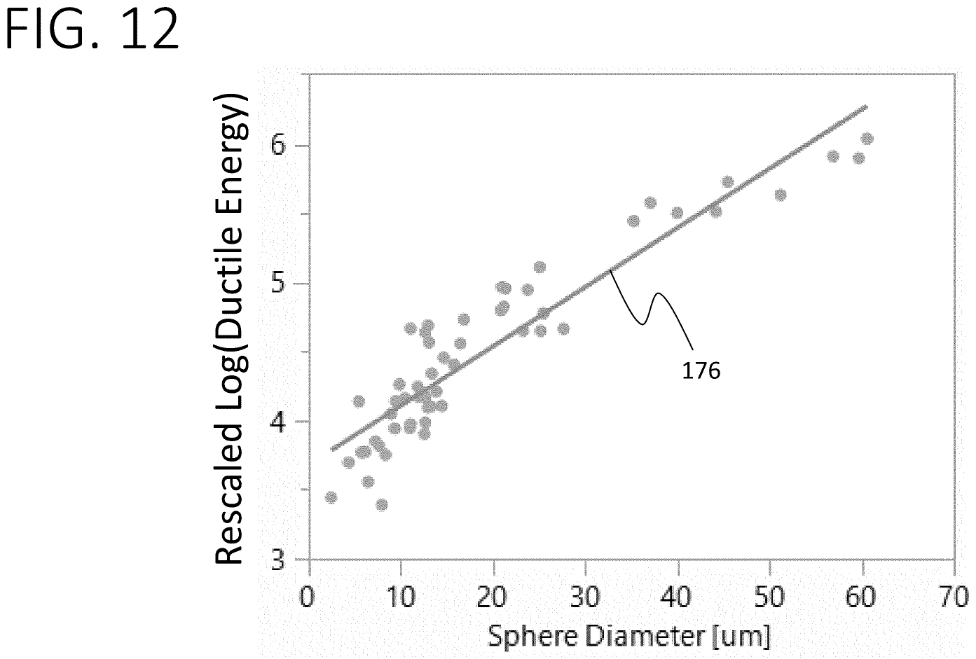

The prediction formula of the Rescaled Log (Ductile Energy) is shown in Equation (1.6). In FIG. 12, the regression line 176 is represented in function of the Rescaled Log (Ductile Energy) experimental data.

Pred Ductile Energyᵢ= [symbol] + [symbol] *x (1.6)

Coefficient [symbol] and [symbol] are determined using Equation (1.7) and (1.8) respectively:

$$= \frac{{SS}_{x y}}{{SS}_{xx}} \qquad ( 1.7 )$$

[symbol]

=y̅− [symbol] x̅ (1.8)

where SSₓₓ and SS_{xy} are defined based on Equation (1.9) and (1.10), respectively:

$$S {S}_{x x} = \sum {x}^{2} - \frac{1}{n} {( \sum x )}^{∧} 2 \qquad ( 1.9 )$$

$$S {S}_{x y} = \sum x y - \frac{1}{n} ( \sum x ) ( ) \qquad ( 1.1 )$$

where x̅ is the mean of all the x-values (where the x values are values of the capsule diameter), y̅ is the mean of all the y-values (where the y values are the Rescaled Log (Ductile Energy) values of single capsule Ductile Energy), and n is the number of capsules measured in the data set.
iv. Volume Fractions

The Volume Fractions (ϕᵢ) of the population are determined via single-particle optical sensing (SPOS), also called optical particle counting (OPC), using the AccuSizer 780 AD instrument or equivalent and the accompanying software CW788 version 1.82 (Particle Sizing Systems, Santa Barbara, California, U.S.A.), or equivalent. The instrument is configured with the following conditions and selections: Flow Rate=1 mL/sec; Lower Size Threshold=0.50 μm; Sensor Model Number=LE400-05SE or equivalent; Auto-dilution=On; Collection time=60 sec; Number channels=512; Vessel fluid volume=50 ml; Max coincidence=9200. The measurement is initiated by putting the sensor into a cold state by flushing with water until background counts are less than 100. A sample of delivery capsules in suspension is introduced, and its density of capsules adjusted with DI water as necessary via autodilution to result in capsule counts of at most 9200 per mL. During a time period of 60 seconds the suspension is analyzed. The range of size used was from 1 μm to 493.3 μm.

### Volume Distribution:

$$CoVv ( \% ) = \frac{{σ}_{v}}{{μ}_{v}} * 100 \\ {σ}_{v} = {\sum }_{i = 1 um}^{493.3 um} ( {x}_{i , v} * {( {d}_{i} - {μ}_{v} )}^{2} ) 0.5 \\ {μ}_{v} = \frac{{\sum }_{i = 1 um}^{493.3 um} ( {x}_{i , v} * {d}_{i} )}{{\sum }_{i = 1 um}^{493 \text{.3} um} {x}_{i , v}}$$

where:
CoVᵥ=Coefficient of variation of the volume weighted size distribution
σᵥ=Standard deviation of volume-weighted size distribution
μᵥ=mean of volume-weighted size distribution
dᵢ=diameter in fraction i
x_{i,v}=frequency in fraction i (corresponding to diameter i) of volume-weighted size distribution

$${x}_{i , v} = \frac{{x}_{i , n} * {d}_{i}^{3}}{{\sum }_{i = 1 um}^{493.3 um} ( {x}_{i , n} * {d}_{i}^{3} )}$$

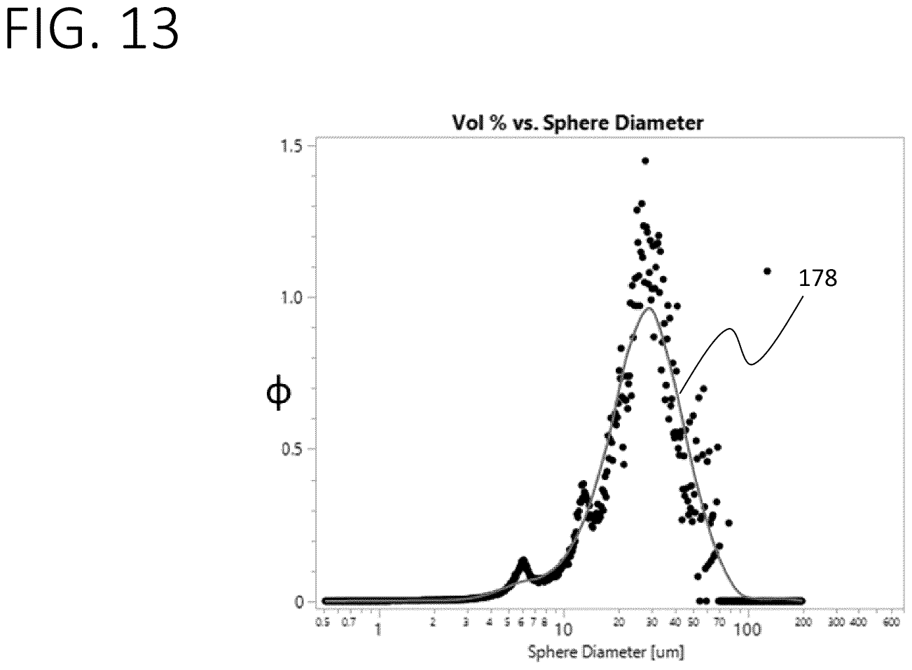

In FIG. 13, the volume fraction is plotted as a function of the particle diameters for the illustrative population, resulting in a distribution 178.

v. Volume-Weighted Ductile Energy

Lastly, the Volume-Weighted Ductile Energy is determined as the summation of the values provided by the Rescaled Log (Ductile Energy) predictions provided at each diameter i weighted with the corresponding volume fraction ϕᵢ as shown by Equation (1.11):

$$Volume Weighted Ductile Energy = \frac{{\sum }_{{d}_{min}}^{{d}_{max}} {ϕ}_{i} Pred Rescaled {Log ( Ductile Energy )}_{i}}{100} \qquad ( 1.11 )$$

where dₘᵢₙ equals 1 μm, while dₘₐₓ equals to 493 μm.

### Determination of a Polymer's Molecular Weight and Related Parameters

The following method describing gel permeation chromatograph with multi-angle light scatter and refractive index detection (GPC-MALS/RI) is used to find molecular weight distribution measurements and related values of the polymers described herein.

Gel Permeation Chromatography (GPC) with Multi-Angle Light Scattering (MALS) and Refractive Index (RI) Detection (GPC-MALS/RI) permits the measurement of absolute molecular weight of a polymer without the need for column calibration methods or standards. The GPC system allows molecules to be separated as a function of their molecular size. MALS and RI allow information to be obtained on the number average (Mn) and weight average (Mw) molecular weight.

The Mw distribution of water-soluble polymers like chitosan is typically measured by using a Liquid Chromatography system (e.g., Agilent 1260 Infinity pump system with OpenLab Chemstation software, Agilent Technology, Santa Clara, CA, USA) and a column set (e.g., 2 Tosoh TSKgel G6000WP 7.8×300 mm 13μm pore size, guard column A0022 6 mm×40 mm PW xl-cp, King of Prussia, PA) which is operated at 40° C. The mobile phase is 0.1M sodium nitrate in water containing 0.02% sodium azide and 0.2% acetic acid. The mobile phase solvent is pumped at a flow rate of 1 mL/min, isocratically. A multiangle light scattering (18-Angle MALS) detector DAWN® and a differential refractive index (RI) detector (Wyatt Technology of Santa Barbara, Calif., USA) controlled by Wyatt Astra® software v8.0 are used.

A sample is typically prepared by dissolving chitosan materials in the mobile phase at ˜1 mg per ml and by mixing the solution for overnight hydration at room temperature. The sample is filtered through a 0.8 μm Versapor membrane filter (PALL, Life Sciences, NY, USA) into the LC autosampler vial using a 3-ml syringe before the GPC analysis.

A dn/dc value (differential change of refractive index with concentration, 0.15) is used for the number average molecular weight (Mn), weight average molecular weight (Mw), Z-average molecular weight (Mz), molecular weight of the peak maxima (Mp), and polydispersity (Mw/Mn) determination by the Astra detector software.

### Viscosity

Viscosity of liquid finished product is measured using an AR 550 rheometer/viscometer from TA instruments (New Castle, DE, USA), using parallel steel plates of 40 mm diameter and a gap size of 500 μm. The high shear viscosity at 20 s⁻¹ and low shear viscosity at 0.05 s⁻¹ is obtained from a logarithmic shear rate sweep from 0.01 s⁻¹ to 25 s⁻¹ in 3 minutes time at 21° C.

Test Method for Determining logP

The value of the log of the Octanol/Water Partition Coefficient (logP) is computed for each material (e.g., each PRM in the perfume mixture) being tested. The logP of an individual material (e.g., a PRM) is calculated using the Consensus logP Computational Model, version 14.02 (Linux) available from Advanced Chemistry Development Inc. (ACD/Labs) (Toronto, Canada) to provide the unitless logP value. The ACD/Labs' Consensus logP Computational Model is part of the ACD/Labs model suite.

### Volume-Weighted Particle Size and Size Distribution

The volume-weighted particle size distribution is determined via single-particle optical sensing (SPOS), also called optical particle counting (OPC), using the AccuSizer 780 AD instrument and the accompanying software CW788 version 1.82 (Particle Sizing Systems, Santa Barbara, California, U.S.A.), or equivalent. The instrument is configured with the following conditions and selections: Flow Rate=1 ml/sec; Lower Size Threshold=0.50 μm; Sensor Model Number=Sensor Model Number=LE400-05 or equivalent; Autodilution=On; Collection time=60 sec; Number channels=512; Vessel fluid volume=50 ml; Max coincidence=9200 . The measurement is initiated by putting the sensor into a cold state by flushing with water until background counts are less than 100. A sample of delivery capsules in suspension is introduced, and its density of capsules adjusted with DI water as necessary via autodilution to result in capsule counts of at least 9200 per ml. During a time period of 60 seconds the suspension is analyzed. The resulting volume-weighted PSD data are plotted and recorded, and the values of the desired volume-weighted particle size (e.g., the mean, the median/50^{th} percentile, 5^{th} percentile, and/or 90^{th} percentile) are determined.

### Procedure for Determination of % Degradation

To determine % degradation, the procedure set forth in the “OECD Guideline for Testing of Chemicals” 301B CO₂ Evolution (Modified Sturm Test), adopted 17 Jul. 1992, is used. For ease of reference, this test method is referred to herein as test method OECD 301B.

### Fabric Treatment

Miele washing machines were used to treat the fabrics. For each treatment, the washing machine was loaded with 3 kg fabric, comprising 1100 g knitted cotton fabric, 1100 g polyester-cotton fabrics (50/50). Also 18 terry towel cotton tracers are added, which weigh together about 780 g. Prior to the treatment, this load was preconditioned twice with 79 g IEC A Base detergent, which is unperfumed and supplied by WFK Testgewebe GmbH, using the 95° C. short cotton cycle followed by two additional 95° C. washes without detergent.

Prior to the test treatment, the load is preconditioned twice, each time using the 95° C. short cotton cycle with 79 g of unperfumed IEC A Base detergent (ex WFK Testgewebe GmbH), followed by two additional 95° C. washes without detergent.

For the test treatment, the load is washed using a 40° C. short cotton cycle, 1200 rpm spin speed with 79 g IEC A Base detergent, which is added at the start of the wash cycle in the appropriate dispenser. A dosage of 35 g of test fabric treatment composition (e.g., according to Examples) is added in the appropriate dispenser. At the end of the wash cycle, the terry towel tracers are removed from the washing machine and line dried overnight. The next day, expert perfumers perform an olfactive assessment for perfume intensity on the dry terry towel tracers. For comparative purposes a reference treatment is also executed where the same fragrance is used as in the test sample but using polyacrylate capsule as delivery particle. All comparative treatments are washed at the same day and analyzed on the same day

### Method of Olfactive Evaluation

After the fabrics have been treated, expert perfumers perform an olfactive assessment for on the dry fabrics perfume intensity at the DRY touchpoints (Dry Fabric Odor=DFO), at a RUB touchpoint (Rubbed Fabric Odor=RFO; fabrics are dried for one day, smelled for DFO, then manually manipulated by rubbing the fabric against itself and smelled again for RFO), and the scores are averaged. Scores are based on a perfume odor intensity scale from 0 to 100, where 0=no perfume odor, 25-slight perfume odor, 50-moderate perfume odor, 75=strong perfume odor, and 100-extremely strong perfume odor. The “Delta RFO” can be reported, which is the difference between the RFO and the DFO.

### Method to Determine Headspace Concentration Above Treated Dry Fabrics

The cotton tracers are analyzed by a fast headspace GC/MS (gas chromatography mass spectrometry) approach. 4×4 cm aliquots of the terry towel cotton tracers were transferred to 25 ml headspace vials. The fabric samples were equilibrated for 10 minutes@65° C. The headspace above the fabrics was sampled via SPME (50/30μm DVB/Carboxen/PDMS) approach for 5 minutes. The SPME fiber was subsequently on-line thermally desorbed into the GC. The analytes were analyzed by fast GC/MS in full scan mode. Ion extraction of the specific masses of the PRMs was used to calculate the total HS response and perfume headspace composition above the tested legs.

In addition to the test capsules, parallel headspace data can be determined using similar methods but with reference capsules, for example polyacrylate-walled (“PAC”) capsules that are beyond the scope of the present disclosure, such as delivery particles made substantially according to the methods described in US Publication 2011/0268802). The data obtained from the reference capsules can be used as a comparison for the results of the inventive capsules. This may be reported as a ratio (e.g., results of inventive particles to the results of the reference capsules).

In the examples below, data results for different trials may be reported as “RFO Headspace [normalized]),” which is the ratio of the headspace results (e.g., Total HS Response) of a trial to the headspace results of Trial 1 for that test. Hence, Trial 1 is “normalized” to 1.0, and the other results are reported with respect to that normalized level. Below, Trial 1 is used as the baseline because, for example, it employs the lowest level of each impact factor of the particle-making process (e.g., % IPM, % cross-linker in oil phase, etc.).

### EXAMPLES

The examples provided below are intended to be illustrative in nature and are not intended to be limiting.

### Example 1. Illustrative Delivery Particle Synthesis

In the following example, the abbreviations correspond to the materials listed in Table 1.

TABLE 1

| Trade Name | Company/City | Material |
|---|---|---|
| ChitoClear | Primex EHF, Siglufjordur, Iceland | Chitosan |
| (grade: 42100) | | |
| Takenate D-110N | Mitsui Chemicals America, Inc., | Polyisocyanate |
| | Rye Brook, NY | prepolymer |

A chitosan stock solution is prepared as following. A potassium persulfate solution was prepared first by dissolving 1.55 g potassium persulfate into 3287.97 g deionized water at 70° C. 154.90 g chitosan ChitoClear was then dispersed into the potassium persulfate solution while mixing in a jacketed reactor. The pH of the chitosan dispersion is then adjusted to 5.10 using 51.72 g concentrated HCl under agitation. The temperature of the chitosan solution is then increased to 85° C. over 60 minutes and then held at 85° C. for a period of time to hydrolyze and depolymerize the chitosan. The temperature is then reduced to 25° C. after the hydrolyzing step over a period of 90 minutes to obtain the acid and potassium persulfate treated chitosan solution. The pH of the chitosan solution is 5.93.

A water phase is prepared by mixing 422.15 g of the above chitosan stock solution in a jacketed reactor. An oil phase is prepared by mixing 146.63 g perfume and 36.66 g isopropyl myristate together along with 5.55 g Takenate D-110N at room temperature. The oil phase is added to the water phase under high shear milling to obtain an emulsion with desired particle size at 25° C. The emulsion is heated to 40° C. over 30 minutes and then hold for another 60 minutes. The obtained emulsion is then heated to 90° C. in 60 minutes and maintained at this temperature for 8 hours while mixing before cools down to 25° C. in 90 minutes. The formed capsules have a volume weighted median particle size of 14.29 microns.

### Example 2. Illustrative Treatment Composition (Liquid Fabric Enhancer)

The table (Table 2) below provides an illustrative treatment composition according to the present disclosure. Specifically, the table shows the formulation for a liquid fabric enhancer (“LFE”) that is suitable, for example, for usage in the rinse cycle of an automatic washing machine. The composition below is also suitable for use in the Fabric Treatment method provided in the Test Method section above. Delivery particles of the formulation below are delivery particles according to the present disclosure, including those of Example 1.

The delivery particles are present in the test LFE compositions at a level so as to provide approximately 0.2% of encapsulated fragrance, by weight of the LFE composition. The pH of the test LFE compositions is adjusted to be approximately 3.

TABLE 2

| Ingredient | % Active (w/w) |
|---|---|
| DiEster Quat ¹ | 6% |
| Encapsulated perfume oil, provided in | 0.2% |
| Chitosan-Based Delivery Particles | |
| Formic Acid | 0.045% |
| Hydrochloric acid | 0.0075% |
| Sodium Hydroxyethane diphosphonic acid | 0.0071% |
| Structurant (cationic polymer) ² | 0.11% |
| Antifoam (silicone) | 0.004% |
| Water | Balance |
| pH | Approx. 3 |

¹ N,N-di(tallowoyloxyethy)-N,N-dimethylammonium chloride, ex Evonik
² Flosoft FS222, ex SNF

### Example 3. Determination of Ductile Energy

For the following example, a population of delivery particles is made according to Example 1. Fifty delivery particles selected to be representative of the diameter distributions are analyzed for mechanical properties and categorized by particle type according to the method provided in the Test Method section.

Table 3 shows the results of the analysis, including particle diameter, the categorization of the individual capsules based on the rupture profile, the speed of rupture, if any, and the Rescaled Log (Ductile Energy).

TABLE 3

| | | | Speed of Rupture | Speed of rupture | Speed of rupture | |
|---|---|---|---|---|---|---|
| Particle | Diameter | Capsule | (First Rupture) | (Second Rupture) | (Third Rupture) | Rescaled Log |
| No. | [um] | Categorization | [nm/s] | [nm/s] | [nm/s] | (Ductile Energy) |
| 1 | 12.2 | Single Rupture | 2638.5 | — | — | 4.410 |
| 2 | 3.8 | Single Rupture | 2237.6 | — | — | 3.154 |
| 3 | 5 | Single Rupture | 332.2 | — | — | 3.241 |
| 4 | 4.1 | Completely Ductile | — | — | — | 3.438 |
| 5 | 3.2 | Completely Ductile | — | — | — | 3.262 |
| 6 | 3.3 | Completely Ductile | — | — | — | 3.304 |
| 7 | 1.9 | Completely Ductile | — | — | — | 2.962 |
| 8 | 1.8 | Single Rupture | 200.5 | — | — | 2.175 |
| 9 | 11.5 | Single Rupture | 5277.5 | — | — | 4.331 |
| 10 | 5 | Multiple Rupture | 291.1 | 494.3 | — | 3.372 |
| 11 | 2.8 | Completely Ductile | — | — | — | 3.186 |
| 12 | 11.6 | Single Rupture | 6422.6 | — | — | 4.264 |
| 13 | 2.7 | Completely Ductile | — | — | — | 3.122 |
| 14 | 3.5 | Completely Ductile | — | — | — | 3.333 |
| 15 | 14.2 | Multiple Rupture | 1448.5 | 1006.9 | — | 4.522 |
| 16 | 1.1 | Completely Ductile | — | — | — | 2.621 |
| 17 | 2.8 | Single Rupture | 330.4 | — | — | 2.579 |
| 18 | 7.7 | Single Rupture | 2378.6 | — | — | 3.914 |
| 19 | 5.2 | Completely Ductile | — | — | — | 3.560 |
| 20 | 7.6 | Completely Ductile | — | — | — | 3.983 |
| 21 | 4.5 | Completely Ductile | — | — | — | 3.485 |
| 22 | 20.2 | Multiple Rupture | 282.7 | 501.2 | 917.6 | 4.763 |
| 23 | 7.6 | Completely Ductile | — | — | — | 4.052 |
| 24 | 4.7 | Completely Ductile | — | — | — | 3.519 |
| 25 | 1.8 | Completely Ductile | — | — | — | 2.901 |
| 26 | 1.7 | Completely Ductile | — | — | — | 2.734 |
| 27 | 3.3 | Completely Ductile | — | — | — | 3.231 |
| 28 | 25.4 | Completely Ductile | — | — | — | 4.823 |
| 29 | 2.4 | Completely Ductile | — | — | — | 3.098 |
| 30 | 8 | Completely Ductile | — | — | — | 4.170 |
| 31 | 8.9 | Single Rupture | 938.7 | — | — | 4.065 |
| 32 | 6.7 | Completely Ductile | — | — | — | 3.890 |
| 33 | 11.2 | Completely Ductile | — | — | — | 4.452 |
| 34 | 6 | Completely Ductile | — | — | — | 3.769 |
| 35 | 12.3 | Multiple Rupture | 936.8 | 703.6 | — | 4.208 |
| 36 | 10.7 | Completely Ductile | — | — | — | 4.315 |
| 37 | 5.1 | Completely Ductile | — | — | — | 3.627 |
| 38 | 9.1 | Completely Ductile | — | — | — | 4.235 |
| 39 | 10.9 | Completely Ductile | — | — | — | 4.407 |
| 40 | 2.2 | Completely Ductile | — | — | — | 3.041 |
| 41 | 16.4 | Single Rupture | 226.3 | — | — | 4.710 |
| 42 | 4.3 | Completely Ductile | — | — | — | 3.394 |
| 43 | 10.4 | Completely Ductile | — | — | — | 4.332 |
| 44 | 5.2 | Completely Ductile | — | — | — | 3.588 |
| 45 | 8.5 | Completely Ductile | — | — | — | 4.076 |
| 46 | 5.6 | Completely Ductile | — | — | — | 3.724 |
| 47 | 3.9 | Completely Ductile | — | — | — | 3.370 |
| 48 | 4 | Single Rupture | 414.3 | — | — | 3.115 |
| 49 | 10.2 | Single Rupture | 6225.3 | — | — | 4.171 |
| 50 | 15.2 | Completely Ductile | — | — | — | 4.636 |

Based on the categorization of the fifty individual capsules, the number and relative proportion (as a percentage) of each category is provided below in Table 4.

TABLE 4

| Category | No. of Capsules | Proportion [%] |
|---|---|---|
| Complete Ductile | 34 | 68 |
| Single Rupture | 12 | 24 |
| Multiple Ruptures | 4 | 8 |

Based on the protocol described in the test method section, the Volume-Weighted Ductile Energy of the population is 4.64.

### Example 4. Effect of Partitioning Modifier Levels on Ductile Energy and Freshness Performance

In the following experiments, populations of perfume delivery particles are prepared substantially according to Example 1, but with different levels of partitioning modifier, specifically isopropyl myristate (“IPM”). The populations are analyzed for Volume-Weighted Ductile Energy, which is reported in Table 5 below.

Furthermore, the delivery particles are provided to a liquid fabric enhancer, which is then used to treat fabrics according to the Fabric Treatment method provided in the Test Methods section above. The treated fabrics are assessed for Rubbed Fabric Odor (RFO) Headspace performance via the Perfume Headspace method, and also for Delta RFO by expert perfumers. The Headspace data is compared to data from reference PAC capsules, and then normalized to the results of Trial 1. The results are reported in Table 5.

TABLE 5

| | | Vol.-Wt.'d | | |
|---|---|---|---|---|
| | IPM level | Ductile | RFO Headspace | Delta RFO |
| Trial | [%] | Energy | [normalized] | (Perfumer) |
| 1 | 10 | 3.55 | 1x | 0 |
| 2 | 20 | 4.30 | 4.3x | 2.5 |
| 3 | 30 | 4.37 | 3.8x | 1.25 |

According to the data in Table 5, relatively greater amounts of partitioning modifier result in relatively greater Volume-Weighted Ductile Energy values for the delivery particle population.

Furthermore, those particles having relatively greater Volume-Weighted Ductile Energy values provide relatively greater RFO Headspace values on treated fabrics, as well as higher Delta RFO scores according to the expert perfumers.

### Example 5. Effect of Cross-Linker Levels on Ductile Energy and Freshness Performance

In the following experiments, populations of perfume delivery particles are prepared substantially according to Example 1, but with different levels of cross-linking agents in the oil phase, specifically polyisocyanate (Takenate D110). The populations are analyzed for Volume-Weighted Ductile Energy, which is reported in Table 6 below.

Furthermore, the delivery particles are provided to a liquid fabric enhancer, which is then used to treat fabrics according to the Fabric Treatment method provided in the Test Methods section above. The treated fabrics are assessed for Rubbed Fabric Odor (RFO) via the Perfume Headspace method, and also for Delta RFO by expert perfumers. The Headspace data is compared to data from reference PAC capsules, and then normalized to the results of Trial 1. The results are reported in Table 6.

TABLE 6

| | Cross- | | | |
|---|---|---|---|---|
| | linker in | Vol.- Wt.'d | | |
| | Oil Phase | Ductile | RFO Headspace | Delta RFO |
| Trial | [%] | Energy | [normalized] | (Perfumer) |
| 1 | 1.34 | 3.31 | 1x | 0 |
| 2 | 2.14 | 4.30 | 150x | 2.5 |
| 3 | 2.94 | 4.64 | 520x | 6.25 |

According to the data in Table 6, relatively greater amounts of cross-linking agents in the oil phase (e.g., polyisocyanate) result in relatively greater Volume-Weighted Ductile Energy values for the delivery particle population.

Furthermore, those particles having relatively greater Volume-Weighted Ductile Energy values provide relatively greater RFO Headspace values on treated fabrics, as well as higher Delta RFO scores according to the expert perfumers.

### Example 6. Effect of pH on Category Proportions and Freshness Performance

In the following experiments, populations of perfume delivery particles are prepared substantially according to Example 1, but with water phases having different pHs. The milling temperature for the particles in this example is 25° C. The populations are analyzed for the relative proportion of Completely Ductile capsules, which is reported in Table 7 below.

Furthermore, the delivery particles are provided to a liquid fabric enhancer, which is then used to treat fabrics according to the Fabric Treatment method provided in the Test Methods section above. The treated fabrics are assessed for Rubbed Fabric Odor (RFO) via the Perfume Headspace method. The Headspace data is compared to data from reference PAC capsules, and then normalized to the results of Trial 1. The results are reported in Table 7.

TABLE 7

| | | Completely | Single- | Vol.-Wt.'d | RFO Headspace | Delta RFO |
|---|---|---|---|---|---|---|
| Trial | pH | ductile [%] | Rupture [%] | Ductile Energy | [normalized] | (Perfumer) |
| 1 | 5.2 | 12% | 43% | 3.31 | 1x | 0 |
| 2 | 5.6 | 34% | 30% | 4.30 | 21x | 2.5 |
| 3 | 6 | 67% | 24% | 4.64 | 52x | 6.25 |

According to the data in Table 7, the particle populations made with water phases at relatively higher pH result in a relatively greater proportion of capsules that are characterized by Completely Ductile behaviour and a relatively lesser proportion of capsules that are characterized by Single-Rupture behaviour.

Furthermore, the populations having a relatively greater proportion of capsules that are characterized by Completely Ductile behaviour are associated with relatively greater RFO Headspace values on treated fabrics.

### Example 7. Effect of Milling Temperature on Category Proportions and Freshness Performance

In the following experiments, populations of perfume delivery particles are prepared substantially according to Example 1, but having different milling temperatures. The populations are analyzed for the relative proportion of Completely Ductile capsules, which is reported in Tables 8 and 9 below. The water phase used for the formation of the particles of Table 8 is characterized by a pH of 5.6. The water phase used for the formation of the particles of Table 9 is characterized by a pH of 5.2.

Furthermore, the delivery particles are provided to a liquid fabric enhancer, which is then used to treat fabrics according to the Fabric Treatment method provided in the Test Methods section above. The treated fabrics are assessed for Rubbed Fabric Odor (RFO) via the Perfume Headspace method. The Headspace data is compared to data from reference PAC capsules, and then normalized to the results of Trial 1. The results are reported in Tables 8 and 9.

TABLE 8

| | Milling | Complete | Single- | Vol.-Wt.'d | RFO Headspace | Delta RFO |
|---|---|---|---|---|---|---|
| Trial | temperature [° C.] | ductile [%] | Rupture [%] | Ductile Energy | [normalized] | (Perfumer) |
| 1 | 15 | 26 | 42.8% | 3.24 | 1x | 0 |
| 2 | 25 | 34 | 30.4% | 4.31 | 21x | 2.5 |

According to the data in Table 8, the particle population made with relatively higher milling temperatures result in a relatively greater proportion of capsules that are characterized by Completely Ductile behavior, a relatively lesser proportion of capsules that are characterized by Single-Rupture behavior, and a relatively higher Volume-Weighted Ductile Energy.

Furthermore, the population having a relatively greater proportion of particles that are characterized by Completely Ductile behavior (and relatively greater Volume-Weighted Ductile Energy) are associated with relatively greater RFO Headspace values on treated fabrics.

TABLE 9

| | Milling | Complete | Single- | Vol.-Wt.'d | RFO Headspace | Delta |
|---|---|---|---|---|---|---|
| Trial | temperature [° C.] | ductile [%] | Rupture [%] | Ductile Energy | [normalized] | Perfumer |
| 1 | 15 | 12.7% | 36 | 3.55 | 1x | 0 |
| 2 | 25 | 7.35% | 22 | 4.37 | 3.3x | 1.25 |

According to the data in Table 9, the particle population made with relatively higher milling temperatures result in a relatively lesser proportion of capsules that are characterized by Single-Rupture behavior, and a relatively higher Volume-Weighted Ductile Energy. Of note, the particles of Trial 2 show a relatively lower proportion of Completely Ductile particles compared to Trial 1; it is believed that this change is associated with the relatively low pH of the aqueous phase.

Furthermore, the population having a relatively lesser proportion of particles that are characterized by Single-Rupture behavior (and relatively greater Volume-Weighted Ductile Energy) are associated with relatively greater RFO Headspace values on treated fabrics.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A treatment composition comprising:
a treatment adjunct, and
a population of delivery particles,
wherein the delivery particles comprise a core and a shell surrounding the core,
wherein the core comprises a benefit agent,
wherein the shell comprises a polymeric material,
wherein the polymeric material comprises the reaction product of a biopolymer and a cross-linking agent,
wherein the population of delivery particles is characterized by at least one, preferably at least two, of the following:
(a) a Volume-Weighted Ductile Energy greater than about 3.5, based on fifty delivery particles selected at random being compressed by a blunt probe moving at 2 μm/s;
(b) at least about 30%, by number, of the delivery particles are characterized as Completely Ductile particles, based on fifty delivery particles selected at random being compressed by a blunt probe moving at 2 μm/s;
(c) less than 35%, by number, of the delivery particles are characterized as Single Rupture particles, based on fifty delivery particles selected at random being compressed by a blunt probe moving at 2 μm/s.

2. The treatment composition according to claim 1, wherein the population of delivery particles is characterized by a Volume-Weighted Ductile Energy of from about 3.5 to about 10.0, based on fifty delivery particles selected at random being compressed by a blunt probe moving at 2 μm/s.

3. The treatment composition according to claim 1, wherein the population of delivery particles is characterized by having at least about 50%, by number, of the delivery particles being characterized as Completely Ductile particles, based on fifty delivery particles selected at random being compressed by a blunt probe moving at 2 μm/s.

4. The treatment composition according to claim 1, wherein the population of delivery particles is characterized by having less than 25%, by number, of the delivery particles being characterized as Single Rupture particles, based on fifty delivery particles selected at random being compressed by a blunt probe moving at 2 μm/s.

5. The treatment composition according to claim 1, wherein the biopolymer is selected from the group consisting of a polysaccharide, a protein, a nucleic acid, a polyphenolic compound, derivatives thereof, and combinations thereof.

6. The treatment composition according to claim 1, wherein the biopolymer is selected from the group consisting of chitosan, starch, modified starch, dextran, maltodextrin, dextrin, cellulose, modified cellulose, hemicellulose, chitin, alginate, lignin, gum, pectin, fructan, carrageenan, agar, pullulan, suberin, cutin, cutan, melanin, silk fibroin, gelatin, collagen, casein, sericin, fibroin, whey protein, zein, soy protein, plant storage protein, gluten, peptide, actin, polynucleotides, RNA, DNA, tannins, lignans, derivatives thereof, and combinations thereof.

7. The treatment composition according to claim 1, wherein the biopolymer is chitosan, a derivative thereof, or a combination thereof.

8. The treatment composition according to claim 1, wherein the biopolymer is characterized by a molecular weight of from about 1 kDal to about 1000 kDal.

9. The treatment composition according to claim 1, wherein the cross-linking agent is a material selected from the group consisting of a polyisocyanate, a polyacrylate, a poly(meth)acrylate, a polyisothiocyanate, an aldehyde, an epoxy compound, a polyphenol, a carbonyl halide, an aziridine, and combinations thereof.

10. The treatment composition according to claim 1, wherein the reaction product is formed in a reaction in which the biopolymer is initially present in an aqueous phase and the cross-linking agent is initially present in an oil phase,
wherein the cross-linking agent is present in the oil phase at a level of from about 1% to about 20%, by weight of the oil phase.

11. The treatment composition according to claim 1, wherein the benefit agent is a fragrance material

12. The treatment composition according to claim 1, wherein the core further comprises a partitioning modifier.

13. The treatment composition according to claim 1, wherein the population of delivery particles are made by a process that includes at least one milling step, wherein the at least one milling step occurs at a temperature of at least about 15° C.

14. The treatment composition according to claim 1, wherein the delivery particles are characterized by a volume-weighted median particle size from about 1 to about 100 microns.

15. The treatment composition according to claim 1, wherein the shells of the delivery particles degrade at least 60% in 60 days when tested according to test method OECD 301B.

16. The treatment composition according to claim 1, wherein the treatment adjunct is selected from the group consisting of surfactants, conditioning actives, deposition aids, rheology modifiers or structurants, bleach systems, stabilizers, builders, chelating agents, dye transfer inhibiting agents, dispersants, enzymes, enzyme stabilizers, catalytic metal complexes, polymeric dispersing agents, clay and soil removal/anti-redeposition agents, brighteners, suds suppressors, silicones, hueing agents, aesthetic dyes, neat perfume, additional perfume delivery systems, structure elasticizing agents, carriers, hydrotropes, processing aids, anti-agglomeration agents, coatings, formaldehyde scavengers, pigments, and mixtures thereof.

17. The treatment composition according to claim 1, wherein the treatment adjunct comprises anionic surfactant, a cationic conditioning agent, or a mixture thereof.

18. The treatment composition according to claim 1, wherein the treatment composition is a fabric care composition, a hard surface cleaner composition, a dish care composition, a hair care composition, a body cleansing composition, or a mixture thereof.

19. The treatment composition according to claim 1, wherein the treatment composition is in the form of a liquid composition, a granular composition, a hydrocolloid, a single-compartment pouch, a multi-compartment pouch, a dissolvable sheet, a pastille or bead, a fibrous article, a tablet, a stick, a bar, a flake, a foam/mousse, a non-woven sheet, or a mixture thereof.

20. The treatment composition according to claim 1, wherein the treatment composition comprises from about 50% to about 99%, by weight of the treatment composition, of water.
